(19) United States
(12) Patent Application Publication
(10) Pub. No.: US 2026/0077113 A1
(43) Pub. Date: Mar. 19, 2026

(54) Introducer Assembly, Drain Placement System, and Related Devices and Methods

(71) Applicant: Surgi Right, LLC, Miami, FL (US)

(72) Inventors: Mauricio Thomas Hernandez, Miami, FL (US); Mauricio Genaro Hernandez, Miami, FL (US); Riicardo Alexander Gomez, Lighthouse Point, FL (US); Juan Carlos Diaz, Coconut Creek, FL (US)

(73) Assignee: Surgi Right, LLC, Miami, FL (US)

(21) Appl. No.: 18/404,877

(22) Filed: Jan. 4, 2024

Related U.S. Application Data
(60) Provisional application No. 63/437,065, filed on Jan. 4, 2023.

(51) Int. Cl. A61M 1/00

(52) U.S. Cl. CPC A61M 1/84; A61M 2202/0014; A61M 2202/04; A61M 2205/586

(57) ABSTRACT

An introducer assembly is provided that can install a drain as an outpatient procedure and corresponding method. The assembly includes an elongate introducer having a distal end with a blunt tip and an opposite proximal end enabling user manipulation. The elongate introducer includes a stylet and a sleeve, one of which forms the blunt tip. The sleeve has a canular wall defining a hollow interior and a stylet access opening therethrough at the sleeve proximal end region. The stylet is arranged as a sleeve insert having a handle laterally extending through the stylet access opening while within the sleeve, which can be slidably withdrawn from the sleeve at the distal end region while remaining portions of the elongate introducer are within a wound to be drained. The elongate introducer can include an embedded scalpel blade and retractable sheath for cutting wound entry and exit incisions when installing a drain.

### CROSS-REFERENCES TO RELATED APPLICATIONS

This application claims priority to U.S. provisional patent application No. 63/437,065 filed on Jan. 4, 2023 entitled, “Introducer Assembly, Drain Placement System and Related Devices and Methods,” which is hereby specifically incorporated by reference in its entirety.

### BACKGROUND

The present application and related subject matter discussed herein relate to medical tools and procedures and particularly to surgical instruments, and more particularly to medical tools and procedures for drain placement including outpatient drain placement tools and procedures.

Postoperative fluid collections (ex: seroma, hematoma, abscess) are common complications after removal of masses from various body sites including abdominal wall, breast, axillary, and inguinal regions. They often occur after lumpectomy, removal of large truncal masses, or after inguinal surgery. As such, drainage treatment needs are constantly in high demand as a necessary part of postoperative care for patients. However, challenges and shortcomings exist for meeting these needs with conventional drainage solutions including complications related to resource utilization and shortcoming with existing medical devices and equipment.

Current drainage solutions are centered on heavy resource utilization. A patient may develop a postoperative fluid collection, schedule an office visit for follow up, and have the fluid either drained with a syringe in the office or scheduled for drain placement under sedation in the operating room (O.R.). For recurrent seromas or larger collections of fluid, patients often require multiple outpatient syringe drainage sessions due to drainage limitations beyond close proximity to the needle tip. Repeat in-office syringe drainage sessions are time-consuming and uncomfortable for the patient, but are generally cost effective, low risk and much less resource intensive compared with more effective O.R. options for drain placement.

If patients require drain placement in the O.R., they must then attend an appointment for preoperative/preanesthesia clearance. They must further adhere to strict nil per os (N.P.O. meaning ‘not by mouth’ preoperative limitations) guidelines, take a day off from work, arrange transportation, register in the hospital, and undergo sedation in the O.R. with an incision and drain placement. Placement of the drain in the O.R. usually requires the use of surgical instruments, such as a hemostat, to pull the drain through the cavity from within the wound along with required O.R. monitoring and life-saving equipment for sedation. Due to the additional steps, costs can be much higher when involving the perioperative hospital system when compared to a simple office visit. The same principles apply for abscesses, which generally require frequent uncomfortable packings, up to several times per week; and for large/persistent hematomas that are fluctuant and often require syringe drainage in the office and/or O.R. utilization for drain placement.

Further, drawbacks and shortcomings exist for existing medical tools and devices used in O.R. drain placement. Current catheters (ex: Jackson-Pratt [JP] drain, Blake drain, Penrose drain) are difficult to place with conventional surgical tools without the aid of O.R. tools. Unnecessary emergency department (E.D.) and O.R. visits are costly, both to the patient, insurance companies, and to the healthcare system. As such, the current approach places undue pressure and further time constraints on a system that is already overwhelmed. Multiple O.R. visits also means repeated exposure to anesthetics and increased risks related to invasive surgical procedures under sedation. Multiple procedures can lead to non-infected seromas becoming infected, which leads to additional complications and increased costs. These challenges are further exacerbated under unexpected circumstances and limitations, such as pandemic or psuedo-pandemic conditions that can preclude O.R. access for procedures deemed elective in view of available alternatives (e.g., multiple outpatient treatments).

Accordingly, there is a need for improved postoperative fluid collection devices, systems and methods including efficient and effective drainage devices, systems and methods for outpatient drain placement, and that can readily be employed by a wide range of practitioners including surgeons, general practice or family physicians, nurse practitioners and other appropriately skilled medical practitioners, as well as improved drain placement devices and methods for O.R. or outpatient procedures.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

One general aspect includes an introducer assembly for tunneling through a wound and thereby guiding a catheter through the wound for placement of a medical device (e.g., drain), which can enable simple and efficient placement of the medical device in an outpatient or surgical environment including placement of drain within a wound. The introducer assembly includes an elongate introducer having a distal end, an opposite proximal end and a middle region therebetween, which includes a sleeve, a stylet insert within the sleeve and removable therefrom during use, and a blunt tip formed by one of the stylet or the sleeve. The stylet includes a stylet body longitudinally extending from a distal end region to an opposite proximal end region, and a handle at the proximal end region laterally extending from the stylet body. The sleeve has a distal end region, a proximal end region, and a sleeve body longitudinally extending therebetween. The sleeve body has a canular wall defining a hollow interior extending from at least a portion of the proximal end region to the distal end region, and defines a stylet access opening into the hollow interior through a side portion of the canular wall at the proximal end region that is configured to receive the stylet body for slidable insertion into and out of the hollow interior. The blunt tip is formed from one of a sleeve blunt tip defined at the distal end region of the sleeve and a stylet blunt tip defined at the distal end region of the stylet extending through an axial opening defined at the distal end region of the sleeve when the stylet body is installed within the hollow interior of the sleeve. The stylet body is installed within the hollow interior of the sleeve for the assembly, such that the proximal end of the elongate introducer is configured to enable manipulation when gripped by a single hand of the user for inserting the blunt tip and a portion of the middle region of the introducer assembly into and through a wound of a patient, the handle extends laterally outward through the stylet access opening, and the handle is configured to enable removal of the stylet body from the hollow interior through the stylet access opening when gripped by a user while the middle region of the portion of the middle region of the elongate introducer is within the wound.

Implementations can include one or more of the following features. The introducer assembly where the handle forms a control tab configured to enhance user control and manipulation of the elongate introducer when gripped by the single hand. The control tab can include: a first side surface, a second side surface opposite the first side surface, and an edge portion between the first side surface and the second side surface, such that the first side surface and the second side surface are oriented generally parallel with a longitudinal axis of the stylet body, a central region of each of the first side surface and the second side surface are laterally offset from the longitudinal axis, and the control tab is configured for being gripped between a thumb and a finger of the user's single hand. The control tab can be generally disc-shaped, such that the first side surface and the second side surface each define a circular shape. A first pivot feature can be formed on the first side surface and a second pivot feature can be formed on the second side surface opposite the first side surface. The first pivot feature can include a first circular rib and the second pivot feature can include a second circular rib. The first circular rib can include a plurality of first concentric ribs, and the second circular rib can include a plurality of second concentric ribs. Further, the first pivot feature can include a first projection extending from the first side surface and the second pivot feature can include a second projection extending from the second side surface.

In some implementations, the sleeve can include a rear extension at the proximal end region longitudinally extending rearward in a proximal direction away from the stylet opening. In some implementations, a longitudinal axis of the sleeve can be contoured between the distal end region and the proximal end region, such that an obtuse angle is defined between the longitudinal axis at the proximal end region and the distal end region of the sleeve. The sleeve body can be malleable permitting user adjustment of the contoured sleeve body and the obtuse angle, and the stylet body can be configured to flex according to the user adjustment of the contoured sleeve body. In some implementations, the sleeve can include a proximal end and the cannular wall at the proximal end can define one of: an axial opening into the hollow interior through the proximal end, and a closed end where the hollow interior is sealed at the proximal end.

The introducer assembly further can include a flexible catheter having a distal end defining an axial opening, an opposite proximal end defining an axial opening, and an elongate tubular body defining a longitudinal pathway between the axial opening at the distal end and the axial opening at the proximal end, in which the distal end is coaxially attached to the sleeve at the proximal end. After withdrawal of the stylet body from the hollow interior, the sleeve can be advanced through the wound and thereby pull the flexible catheter into the wound. After withdrawal of the stylet body from the hollow interior, the sleeve and a distal portion of the flexible catheter can be pulled out of the wound and the patient to advance the fenestrated drain into the wound, and the distal portion of the flexible catheter can be disconnected from the sleeve to attach to a drain collector for wound drainage.

In some implementations, the blunt tip is formed from the stylet blunt tip defined at the distal end region of the stylet extending through the axial opening defined at the distal end region of the sleeve when the stylet body is installed within the hollow interior of the sleeve, and the stylet body further includes a slot oriented in the longitudinal direction of the stylet body defined in the distal end region of the stylet body and through the blunt tip and a scalpel blade embedded in the stylet body within the slot and located outside the sleeve proximate the axial opening at the distal end region of the sleeve. The scalpel blade can be arranged to cut an entry incision through the user's skin for gaining entry into the wound and to cut an exit incision through the user's skin for withdrawing the sleeve from the wound. As such, the distal end region of the stylet body, the blunt tip, and the slot form a protective sheath about the scalpel blade. The protective sheath can be arranged to retract proximally into the sleeve for exposing the scalpel blade and cutting the entry incision and/or the exit incision.

In some implementations, the sheath can be selectively retractable by the user, such that the handle has a first length in the longitudinal direction of the stylet, and the stylet access opening has a second length in the longitudinal direction larger than the first length. The stylet body and the sheath can be biased forward in the distal direction and translatable rearward in the proximal direction within the hollow interior of the sleeve. The handle can be configured to enable the user to slide the handle rearward in the proximal direction within the stylet access opening and thereby translate the stylet body and the sheath to the retracted position for exposing the scalpel blade. In some implementations, the sheath can be configured to retract automatically in response to contact interference with skin of the patient when forming the entry incision and the exit incision. In such an arrangement, the stylet body can include a coaxial channel defined through the stylet body extending between the rear proximal side of the scalpel blade to the proximal end region, a retraction gap defined at a middle region of the stylet body, and a bias member disposed within the retraction gap distally biasing the distal end region of the style body longitudinally away from the proximal end region of the stylet body. The stylet body can further include a scalpel support extending from a rear proximal side of the scalpel blade through the coaxial channel, the retraction gap and the bias member to the proximal end region of the stylet body, in which the scalpel support is fixedly attached to the proximal end region stylet body and the sheath is configured to translate about the scalpel support. In some implementations, the sleeve can be tapered between the distal end region and the proximal end region. As such, the sleeve can include a first cross-sectional area at the distal end region and a second cross-sectional area at the proximal end region that is larger than the first cross-sectional area.

Some aspects can include an outpatient method for draining fluids from a wound of a patient using the introducer assembly. The method can include obtaining the introducer assembly, creating an input incision through the skin of the patient proximate the wound, and inserting through the input incision the blunt tip and a portion of the middle region of the elongate introducer. The method can further include advancing the elongate introducer within the wound along a desired drain path from the input incision to a desired location for an exit incision while keeping the side grip outside the wound and, optionally, advancing the blunt tip within the wound against the patient's skin to identify a location for an exit incision. The method can further include creating the exit incision, advancing the blunt tip through the exit incision, withdrawing the stylet body from the hollow interior of the sleeve through the lateral access opening of the sleeve disposed outside of the wound, and advancing the sleeve through the exit incision and out of the wound and thereby concurrently pulling a portion of the elongate catheter into the wound through the input incision and out of the wound through the exit incision. The method can also include continuing advancing the elongate catheter through and out of the wound including drawing the fenestrated drain into the wound through the input incision and through the wound until the fenestrated drain is positioned at a desired drainage location, and stitching closed the input incision. The method can further include disconnecting the proximal end region of the sleeve from the distal end of the elongate catheter, connecting the distal end of the elongate catheter to a drain collector, and draining the wound.

In some implementations of the method using the introducer assembly, the blunt tip is formed from the stylet blunt tip extending through the axial opening defined at the distal end region of the sleeve when the stylet body is installed within the hollow interior, the stylet body includes an embedded scalpel blade, and the blunt tip and the distal end region of the stylet body form a retractable sheath configured to retract rearward into the sleeve to expose the embedded scalpel blade. As such, creating the input incision for the method can include retracting the retractable sheath to expose the embedded scalpel blade, and advancing the embedded scalpel blade through the patient's skin to create the input incision. Further, creating the exit incision for the method can include retracting the retractable sheath to expose the embedded scalpel blade, and advancing the embedded scalpel blade through the patient's skin to create the exit incision. The retractable sheath can be selectively retractable by the user, such that for creating the input incision and creating the exit incision, each of the retracting the retractable sheath can include manually retracting the retractable sheath, and advancing the exposed scalpel blade through the user's skin. The retractable sheath can also be automatically retractable in response to interfering contact of the sheath with the user's skin, such that for creating the input incision and creating the exit incision, each of the retracting the retractable sheath can include advancing the elongate introducer for advancing the blunt tip and the sheath into contact with the user's skin, and continuing advancing the elongate introducer for advancing the exposed scalpel into and through the user's skin.

Other medical devices, components for medical devices, assemblies, systems, and/or methods according to novel and inventive aspects, features and example arrangements and implementations will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1A is a plan view of a schematic representation of an example introducer assembly that can be used for drain placement according to aspects, features and concepts described herein.

FIG. 1B is a plan view of the elongate introducer or probe for the introducer assembly of FIG. 1A.

FIGS. 2A to 2F are elevation views showing use of the introducer assembly of FIG. 1A in an outpatient drain placement procedure for draining a collection of fluid from a wound.

FIG. 3A is a plan view of a schematic representation of a further example introducer assembly that can be used for drain placement according to aspects, features and concepts described herein.

FIG. 3B is a plan view of the stylet of the elongate introducer for the introducer assembly of FIG. 3A.

FIG. 3C is a plan view of the sleeve of the elongate introducer for the introducer assembly of FIG. 3A.

FIG. 4A is a plan view of a schematic representation of another example introducer assembly that can be used for drain placement according to aspects, features and concepts described herein.

FIG. 4B is a plan view of the stylet of the elongate introducer for the introducer assembly of FIG. 4A.

FIG. 4C is a plan view of the sleeve of the elongate introducer for the introducer assembly of FIG. 4A.

FIG. 5A is a side perspective view of a schematic representation of a further example introducer assembly that can be used for drain placement according to aspects, features and concepts described herein.

FIG. 5B is a close view of the blunt tip and distal end of the elongate introducer for the introducer assembly of FIG. 5A as indicated in FIG. 5A.

FIG. 6 is an exploded perspective view of the introducer assembly of FIG. 5A.

FIG. 7 is a perspective view of the stylet of the elongate introducer for the introducer assembly of FIG. 5A.

FIG. 8 is a perspective view of the sleeve of the elongate introducer for the introducer assembly of FIG. 5A.

FIG. 9 is a perspective view of the flexible catheter of the introducer assembly of FIG. 5A.

FIG. 10 is a perspective view of the fenestrated drain of the introducer assembly of FIG. 5A.

FIG. 11A is a side perspective view of a schematic representation of another example introducer assembly that can be used for drain placement according to aspects, features and concepts described herein, which depicts example features pertaining to an embedded scalpel and a retractable sheath.

FIG. 11B is a close perspective view of the blunt tip and the distal end of the elongate introducer for the introducer assembly of FIG. 11A shown with the sheath in an unretracted state.

FIG. 11C is a close perspective view of the blunt tip and the distal end of the elongate introducer for the introducer assembly of FIG. 11B shown with the stylet body and the sleeve partially transparent for viewing the scalpel blade and a distal portion of the scalpel support arm embedded therein.

FIG. 12 is an exploded perspective view of the introducer assembly of FIG. 11A.

FIG. 13A is a magnified close perspective view of the blunt tip and the distal end of the elongate introducer for the introducer assembly as depicted in FIG. 11B shown with the sheath in an unretracted state.

FIG. 13B is a magnified close perspective view of the blunt tip and the distal end of the elongate introducer for the introducer assembly of FIG. 13A depicted with the sheath in a retracted state.

FIG. 14 is a perspective view of the stylet of the elongate introducer for the introducer assembly of FIG. 11A.

FIG. 15 is a perspective view of the stylet of the elongate introducer for the introducer assembly of FIG. 11A as depicted in FIG. 14, which is shown with the stylet body partially transparent for exposing the coaxial channel defined within the stylet body, the scalpel blade embedded within the stylet body, and the blade support arm disposed within the coaxial channel and extending proximally from the rear side of the scalpel blade to the proximal end region of the stylet body.

FIG. 16A is a perspective view of the handle and control tab of the stylet body of the elongate introducer for the introducer assembly of FIG. 11A.

FIG. 16B is a perspective view of the scalpel blade and the blade support arm of the stylet body of the elongate introducer for the introducer assembly of FIG. 11A.

FIG. 16C is a perspective view of the bias member depicted as a spring of the stylet body of the elongate introducer for the introducer assembly of FIG. 11A.

FIG. 17A is a side perspective view of the distal end region and the middle region of the stylet body of the elongate introducer for the introducer assembly of FIG. 11A shown without the scalpel blade and blade support arm.

FIG. 17B is an exploded side perspective view of components for the distal end region and the middle region of the stylet body of the elongate introducer for the introducer assembly of FIG. 11A as depicted in FIG. 17A.

FIG. 18A is a magnified close perspective view of the blunt tip and the distal end of the stylet body of the elongate introducer for the introducer assembly of FIG. 11A, which is depicted in the unretracted state that corresponds with the close view of FIG. 13A and shown without the sleeve and with the exterior portions of the stylet body partially transparent for exposing the scalpel blade and a distal portion of the blade support arm embedded within the stylet body.

FIG. 18B is a magnified close perspective view of the blunt tip and the distal end of the stylet body of the elongate introducer for the introducer assembly of FIG. 11A shown in FIG. 18A without partial transparency, and depicted with the sheath in the retracted state.

FIG. 19 is a perspective view of the sleeve of the elongate introducer for the introducer assembly of FIG. 11A.

FIG. 20A is a perspective view of the fenestrated drain of the introducer assembly of FIG. 11A.

FIG. 20B is a perspective view of the flexible catheter of the introducer assembly of FIG. 11A.

FIG. 21 is a side perspective view of a schematic representation of an elongate assembly for yet another example introducer assembly that can be used for drain placement according to aspects, features and concepts described herein, which depicts example features pertaining to an embedded scalpel and a manually retractable sheath.

FIG. 22 is an exploded side perspective view of the elongate introducer for the introducer assembly of FIG. 21.

FIG. 23 is a side perspective view of the elongate introducer for the introducer assembly of FIG. 21 shown with the sleeve, the handle, and outer portions of the stylet body partially transparent for exposing the stylet body installed within the sleeve and components of the stylet body embedded therein including the scalpel blade, the blade support arm, the biasing member, and the scalpel arm stop.

FIG. 24 is schematic representation of a method for installing a drain using an introducer assembly according to aspects, features and concepts described herein.

### DETAILED DESCRIPTION

The example arrangements and schematic representations of medical tools, components and assemblies described herein can advantageously be used with or in combination with a wide variety of surgical tools and devices, and for various medical procedures involving the introduction and placement of a medical device connected to a catheter within the body. In particular, example arrangements and schematic representations of medical tools, components and assemblies described herein can advantageously be used for introducing and placing a drain within the body at a location (i.e., wound) having a collection of fluid requiring drainage, including using the same and performing such procedures under outpatient or operating room conditions.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used herein, a surgical device or tool or clinical instrument refers to a medical instrument having contact surfaces that are configured to engage organs, tissues and/or portions of a surgical cavity or wound to thereby move, hold, lift, retain, suture or otherwise engage, interface or make contact with the target tissue and perform clinical functions as appropriate for the surgical environment. The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to a target tissue would be the distal end of the tool, and the end opposite the distal end (i.e., the end manipulated by the user) would be the proximal end of the tool.

Further, specific words chosen to describe one or more example schematic arrangements, assemblies, parts, components and optional elements or features are not intended to limit the invention. For example, spatially relative terms—such as “beneath”, “below”, “lower”, “above”, “upper”, “proximal”, “distal”, and the like—can be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures were turned over, elements described as “below”, or “beneath” other elements of features would then be “above” or “over” the other elements or features. Thus, the term “below” can encompass both positions and orientations of above and below. A device can be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Aspects, features and concepts described herein generally pertain to medical devices or tools for relatively simple, efficient and effective introduction and placement of a fenestrated drain or other medical device connected to a catheter within a portion of a patient's body, such as within a wound having a collection of fluid, which can be used and performed under O.R. and outpatient or other non-surgical conditions (i.e., under local anesthesia without sedation). As such, significant advantages and benefits can be gained by avoiding unnecessary complications, expenses, and other challenges of conventional procedures for draining fluids, and even further benefits realized through improved device functionality and effectiveness along with procedural advantages enabled according to aspects, features and concepts described herein.

Accordingly, aspects, features and concepts described herein along with schematic depictions of example medical tools, devices, assemblies, components and related procedures and methods, significant functional and procedural advantages for placement of a drain or other device attached to a catheter within the body of a patient. For example, significant manipulation and control benefits can be provided during placement procedures through the use of introducers, introducer assemblies and related devices and components according to aspects and features described herein, which can include relatively simple devices for effectively performing these procedures without requiring complicated control mechanisms or specialized environments (e.g., without operating room type support). These devices can enable precise placement and effective installation of a drain or other medical device within a wound through minimally invasive, accurate mechanisms for tunneling a catheter/drain pathway through the wound between an input incision and an exit incision. Further, significant beneficial options and features can be enabled according to aspects and features of these devices, such as for making necessary incisions and minimizing incision sizes via options for including an embedded scalpel coaxially aligned with the elongate introducer at its proximal end region, and for enhancing manipulation and control of the elongate introducer while tunneling through the wound along a desired placement path via options for a removable handle that can be removed from the proximal end region of the assembly after traversing the wound prior to fully advancing the introducer through the wound.

These and other aspects, features, concepts, advantages and benefits are described in greater detail below along with schematic depictions and representations of example implementations and arrangements for corresponding medical devices and components, and of related methods for effective use of the same.

### Elongate Introducer/Probe, Catheter and Drain Assembly

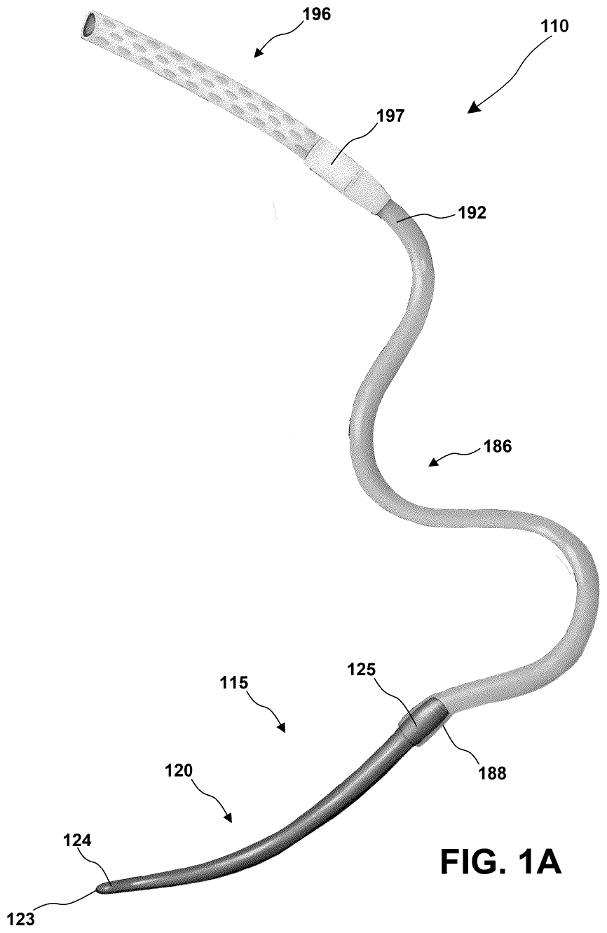

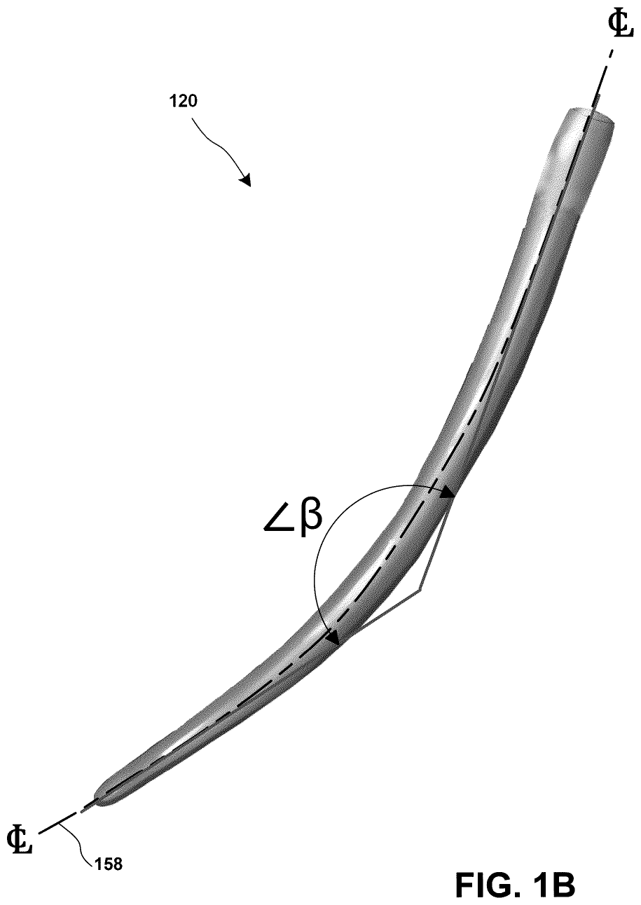

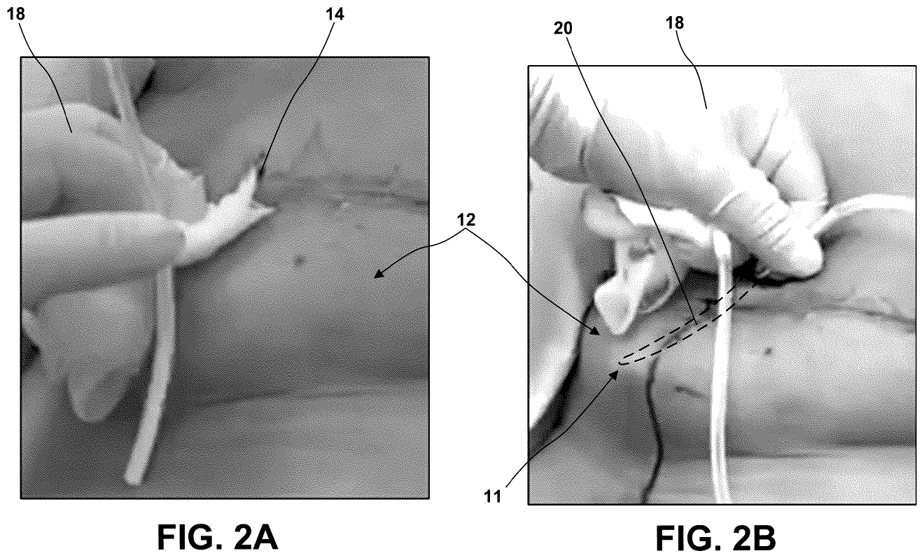

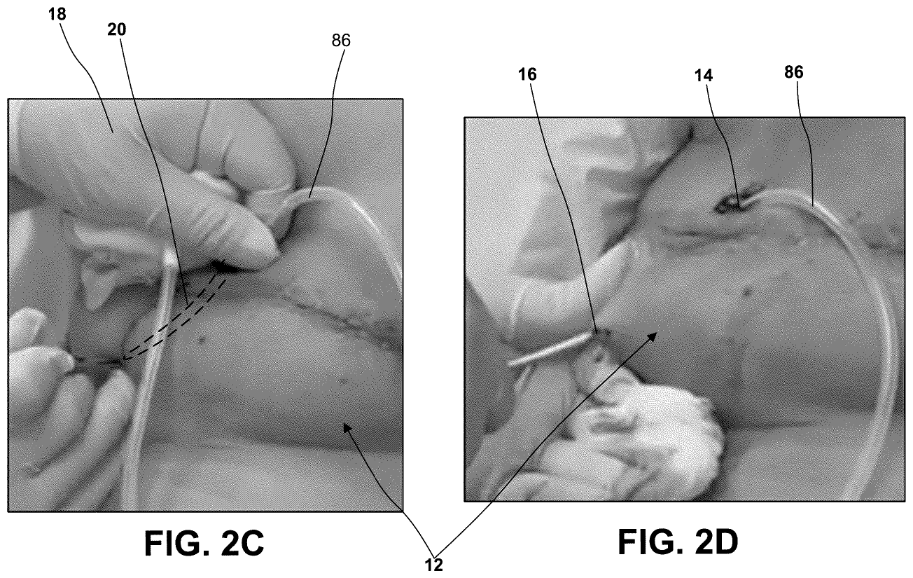

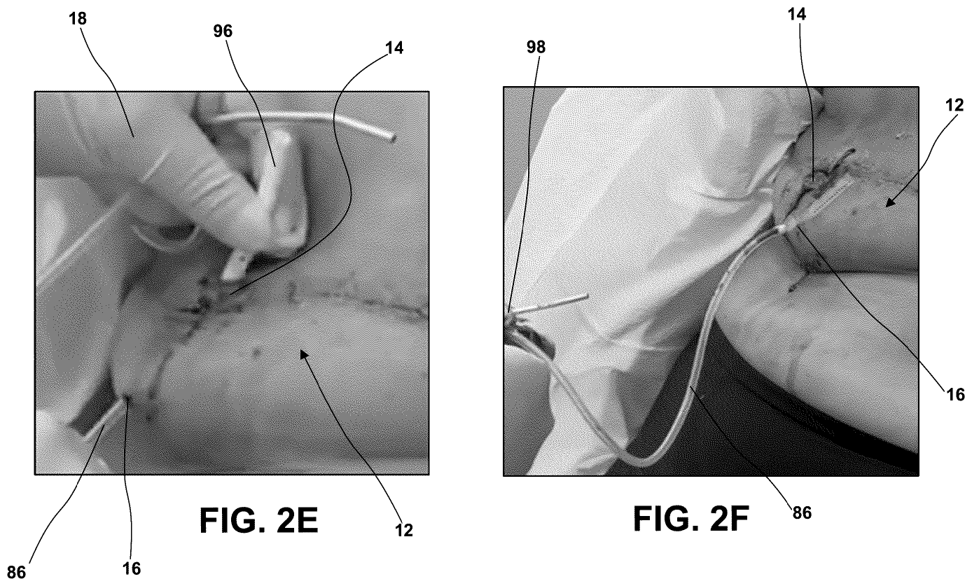

Referring now to FIGS. 1A and 1B along with FIGS. 2A to 2F discussed below that show portions of an outpatient drain placement procedure, a schematic representation of an introducer assembly 110 is generally shown for introducing and placing a drain 196 (or other medical device) within a wound or at another desired location in a patient's body. As used herein, the term “wound” generally refers to target region of a patient's body for intended placement of the drain or other medical device. However, it is understood that the “wound” location is not limited to an area of the patient identified as a postoperative region, a stressed or damaged region, or region that otherwise has been injured or traumatized. Rather, the term “wound” as used herein refers to the target region for a placement procedure and, in particular with respect to examples depicted herein, the target region for placement of a drain within the patient's body.

As shown in FIGS. 1A and 1B, the introducer assembly 110) includes an elongate introducer 115 in the form of a generally rigid blunt tip probe 115 having a distal end region 124, an opposite proximal end region 125, and a body 122 longitudinally extending therebetween, as well as a blunt tip 111, 123 formed on a leading portion of the distal end region. The blunt tip probe 115 can be coaxially attached to a flexible catheter 186, which can be attached to a fenestrated drain 196 for placement in a wound proximate a collection of fluid. The blunt tip probe 115 acts as an introducer the user can effectively manipulate and control for tunneling through the wound along a desired pathway between an input incision and an exit incision and thereafter for pulling the catheter 186 and drain 196 along the pathway to place the drain at the desired location.

The blunt tip probe 115 can be made from a medical grade aluminum or other metal, an elastomer, or other resilient, substantially rigid, medical grade material or combination of materials. The size and diameter of the blunt tip 123 and distal end region 124 can be sized in accordance with an appropriate size for the flexible catheter 186, wound and drainage needs. For example, the blunt tip 123 and diameter at the distal end region 124 can be about 3 mm or other appropriate size for enabling advancement of the introducer assembly within and through soft tissue of the wound with minimal interference or likelihood of causing tissue damage, and the proximal end region can have an outer diameter of about 5 mm or other appropriate size for ensuring secure, inline or coaxial attachment with a flexible catheter having, as an example, an outer diameter of about 5 mm (e.g., 4.7 mm or 5.3 mm catheter corresponding with a catheter of 14 or 16 French units (Fr)).

Further, the blunt tip probe 115 can be tapered for increasing in diameter along its length from a first diameter D1 at the distal end region 124 to a second diameter D2 at its proximal end region 125 that is larger than the first diameter. It is understood that other diameters and sizes can be used as appropriate for an intended usage, drain size and other parameters. Likewise, the incision diameters can be sized to correspond with the diameter, D1, of the blunt tip or distal end region 124 of the blunt tip probe including the diameter for the input incision 14 shown in FIG. 2A and the exit incision 16 shown in FIG. 2D at opposite sides along the wound and pathway for the desired placement and drain placement location. For instance, the input incision 14 and the exit incision 16 can be created as a small, precise incision of about 3 mm corresponding with the diameter of the blunt tip, such as using a #15 blade scalpel.

Matching the incision size with the diameter, D1, at the distal end region 124 in combination with having a tapered blunt tip probe 115 with a larger diameter, D1, at the proximal end region 192, can enable a close fit about the flexible catheter 186 at the exit incision for ensuring an airtight seal, which can further be secured via stitches such as finger-trap sutures. The input incision can be stitched closed after placement of the drain for enabling negative pressure wound drainage.

As best seen in FIG. 1B, the blunt tip probe 115 can be contoured along its length between the distal end region 124 and the proximal end region 125, such that an obtuse angle, P, is formed between the proximal end region and the distal end region. The contour can be formed as depicted in FIG. 1B as a curvature along the length or portions of the length of the blunt tip probe 115. Alternatively, the contour can be formed via one or more discrete bends along the length or portions of the length of the blunt tip probe 115, such that the blunt tip probe is angled or bent between its opposite ends. Further, the blunt tip probe 115 can be malleable to allow user adjustment of the contour between the opposite ends as desired or appropriate for the intended pathway and drain installation location.

The proximal end region of the blunt tip probe 115 is arranged for being gripped by a single hand of the user, such that the contour along the length of the blunt tip probe 115 enables the user to manipulate the blunt tip probe 115 while advancing through the wound along the desired pathway between the input incision and exit incision. The distal end 188 of the flexible catheter can overlap the proximal end region 125 a sufficient distance for enhancing the user's grip and control along with ensuring a secure connection is maintained with the catheter. The distal end 188 can be closed for avoiding contamination of the catheter interior while traversing the wound.

Referring now to FIGS. 2A to 2F along with continued reference to FIGS. 1A and 1B, portions of a relatively simple and efficient outpatient drain placement procedure are shown that were performed using the introducer assembly 110 of FIGS. 1A & 1B with the patient receiving local anesthetic (not shown) without being sedated. Although shown using introducer assembly 110, it is understood that other introducer assemblies and devices according to aspects, features and concepts discussed herein including example schematic introducer assemblies shown and described hereafter can be used for performing similar procedures. As such, aspects and features described and shown along with FIGS. 2A to 2F can pertain to various example introducer assemblies shown and described herein and to other implementations of introducer assemblies according to aspects, features and concepts described herein.

The outpatient drain placement procedure shown in FIGS. 2A to 2F is for placement of a drain 96 within a portion of a postoperative wound 12 having fluid collected therein. FIG. 2A shows wound 12 after the physician created an input incision 14 at a desired location at an upper portion of the wound. FIG. 2B shows the physician manipulating and advancing distal and middle regions of the elongate introducer or blunt tip probe 115 within the wound 12 after insertion through the input incision 14. As shown, the physician can use a single hand 18 to grip the proximal end region of the blunt tip probe/elongate introducer 15 and manipulate it along a desired pathway within the wound based on the contoured shape of the blunt tip probe. Further, the contoured blunt tip probe is configured so that the blunt tip 11 can be guided within the wound and advanced against the patient's skin while inside the wound 12 for visibly identifying to the user a location for the exit incision.

FIG. 2C shows the physician confirming and thereafter marking the location for the exit incision in preparation for cutting (not shown) the exit incision 16 at the desired location. As shown. the proximal end region of the blunt tip probe remains outside of the wound prior to making the exit incision and advancing the blunt tip therethrough. This allows the single hand 18 of the physician continued grip access to the proximal end region for continued advancement of the blunt tip through the exit incision 16, which the user can thereafter grip for pulling the blunt tip probe and catheter through the wound. FIG. 2D shows the blunt tip, distal end region and a portion of the middle region of the blunt tip probe extending through the exit incision along with the proximal end region of the blunt tip probe and a portion of the flexible catheter drawn through the input incision 14. Even though the blunt tip probe is contoured for enhanced control and manipulation thereof in and through the wound by the single hand of the user, the proximal end region of the blunt tip probe 115 forms a low-profile, coaxial connection with the flexible catheter such that the flexible catheter can readily be drawn into and through the input and exit incisions and through the wound 12.

FIG. 2E shows continued advancement of the flexible catheter such that the fenestrated drain attached to the proximal end of the catheter is being drawn through the input incision 14 and into the wound 12 to the desired placement location therein with a distal end portion of the catheter extending through exit incision. FIG. 2F shows the fenestrated drain fully placed at the desired location within the wound 12, the input incision 14 stitched closed, and the flexible catheter in the process of being attached to a vacuum drain collector.

According to additional aspects, features and concepts described herein, an Outpatient Drainage Catheter (ODC) Kit (not shown) can be provided for enabling similar safe and efficient outpatient procedures for drain placement. The kit can be self-contained and, as an example, include the following: A skin preparation (e.g., povidone iodine or alcohol swabs), tape, gauze, a 10 cc vial of 1% lidocaine, a 5 cc syringe, a blunt needle to draw up medication, a 25 G needle for local anesthetic administration, a flexible catheter similar to catheter 188, a fenestrated drain similar to drain 196, and an elongate introducer similar to the blunt tip probe 110 described above and other examples described below or according to aspects, features and concepts described herein. After numbing the skin with ˜3 cc of 1% lidocaine at the anticipated insertion site (point A), a 15 blade with a plastic handle or other appropriate scalpel can be used to create a small incision at the fluctuation point. The blunt-tip probe can then be inserted as described above and tunnel to a different point (point B), where an additional small incision can be made. Through the use of the elongate introducer (e.g., blunt tip probe 115), the catheter can be pulled through and remain inserted in the new cavity along with drain at the desired placement. The ODC Kit (not shown) can come with a single suture (2.0 nylon) that can be used to hold the catheter in place.

Thus, use of an introducer assembly (e.g., blunt tip probe 115) according to aspects, features and concepts described herein including the above examples can enable efficient and effective outpatient procedures, and further enhance operating room procedure options, for accurately placing a wound drain or other device at a desired location. The use of a wound drain for a drainage period, such as for 30 min. to an hour or more, or for as needed repeat wound drainage, can provide significant advantages and benefits as noted above over conventional multiple syringe drainage procedures or far more complex and expensive operating room drain placement procedures. Further, such an arrangement for the introducer assembly can enable accurate control for guiding placement of the wound drain in a minimally invasive manner compared with conventional devices and procedures including those used along with operating room support with the patient under sedation.

Additional advantages including enhanced control and manipulation benefits can incorporated in other arrangements for an introducer assembly, such as control and manipulation benefits enabled through the use of stylet insert with a sleeve to form the elongate introducer or blunt tip probe as discussed hereafter.

### Insert Stylet and Sleeve Assembly

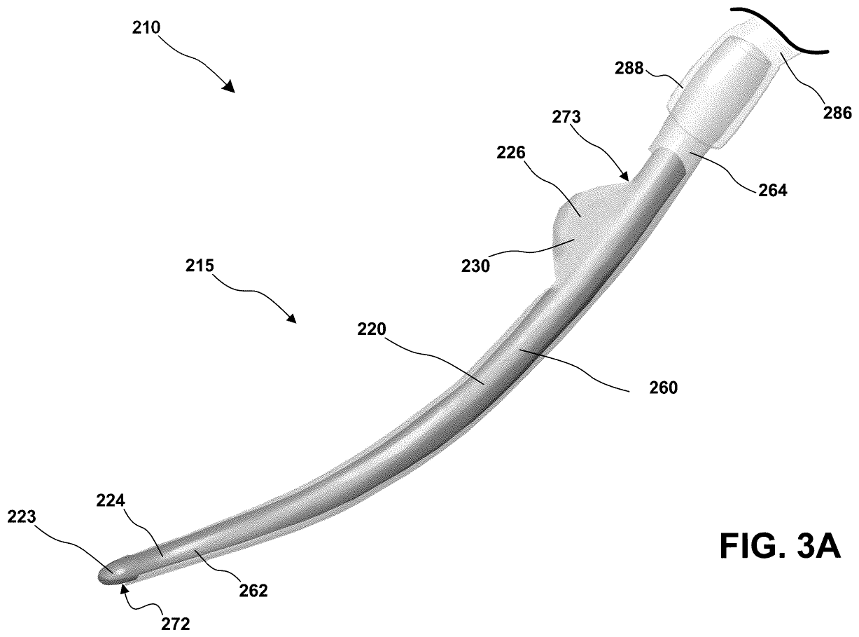

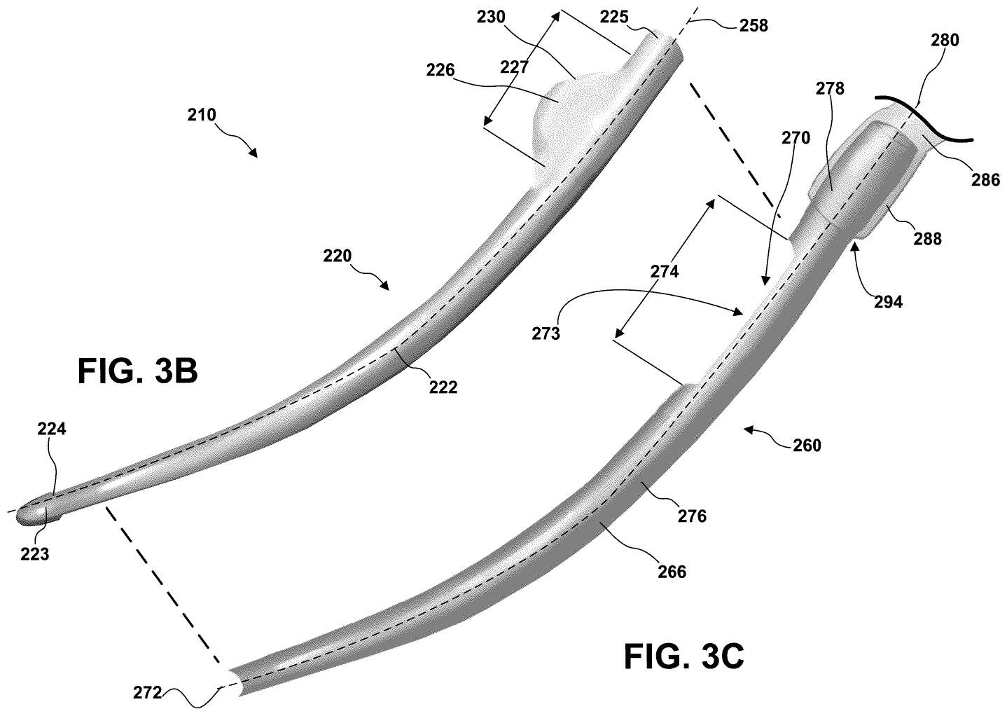

Referring generally to FIGS. 3A to 3C, an introducer assembly 210 is shown generally having the same aspects, features and preferences described above along with introducer assembly 110 except as described hereafter. As such, like numbers refer to like features. Introducer assembly 210 can include a flexible catheter 286 (partially shown) attached to a proximal end of a sleeve member of the elongate introducer as discussed below, and a fenestrated drain (not shown) coaxially attached to the flexible catheter as described above along with FIGS. 1A and 1B.

Introducer assembly 210 generally differs from introducer assembly 110 described above. in that the blunt tip probe or elongate introducer 215 is formed as a removable assembly of two primary components; Namely, a sleeve 260 defining a hollow interior 270, and a stylet 220 arranged as a removable insert within the hollow interior that has a laterally extending handle 226 at a proximal end region 225 of the stylet. The laterally extending handle 226 is arranged to provide enhanced user manipulation and control of the elongate introducer 215 into and through the wound while being advanced along a desired path, which can enable greater accuracy for placement of the drain or other device within the wound. The insert arrangement further allows for easy removal of the stylet insert from within the sleeve prior to the sleeve fully advancing into the wound and pulling the attached flexible catheter. Such an arrangement permits smooth entry of the sleeve, the attached flexible catheter, and the fenestrated drain connected to the catheter into and through the wound without interference from the handle that can be gripped between a thumb and finger of the user for easily withdrawing the stylet from within the sleeve prior to entry of the distal end of the elongate introducer into the wound, along with providing enhanced manipulation and control based on availability of the handle while traversing a desired pathway within the wound.

For example, the outpatient procedure shown in FIG. 2B using an example elongate introducer similar to the elongate introducer 115 shown in FIGS. 1A and 1B shows the user gripping the proximal end region of the elongate introducer between the thumb and a finger of the user's hand 18 without having any shape features or other grip controls for assisting with rotation of the elongate introducer between the thumb and finger. The laterally extending handle 226 can act as a control tab 230 that can enhance the user's maneuverability and orientation controls for the elongate introducer 215 while attempting to advance the same along a desired path through the wound 12.

The blunt tip probe or elongate introducer 215 includes a stylet 220, a sleeve 260, and a blunt tip 211 defined at a distal end of the elongate introducer. The stylet includes a stylet body 222 longitudinally extending from a distal end region 224 to an opposite proximal end region 225, and a handle 226 at the proximal end region of the stylet body laterally extending therefrom. The sleeve 260 has a distal end region 262, a proximal end region 264, and a sleeve body 266 longitudinally extending therebetween. The sleeve body 266 has a canular wall 268 defining a hollow interior 270 extending from at least a portion of the proximal end region to the distal end region. A stylet access opening 273 into the hollow interior 270 is defined through a side portion of the canular wall 268 at the proximal end region 264, which is sized and arranged to receive the stylet body 222 for slidable insertion into and out of the hollow interior 270. The blunt tip 211 is formed from a stylet blunt tip 223 at the distal end region 224 of the stylet 220 that extends through an axial opening 272 defined through the canular wall 268 at the distal end region 262 of the sleeve 260.

Similar to elongate introducer 115 as described above, the stylet 220 and the sleeve 260 can each be formed from a suitable medical grade material including metal, such as aluminum, plastic including various polymers, and combinations of the same. The sleeve 260 can be formed as a substantially rigid component, but can also be malleable for permitting user shape customization as appropriate, such as a contour or bend adjustments along the length of the sleeve in accordance with intended usage. The stylet 220 can be formed from a more flexible material than the sleeve for enabling easy removal from the hollow interior 270 through the stylet access opening 273 prior to fully inserting the sleeve into the input incision and wound, as well as for initial installation into the hollow interior if not installed therein in advance.

Notably, conventional stylet usage and arrangements including stylet arrangements in which the stylet is arranged as an insert within a sleeve or other elongate member teach and are configured in an opposite manner. In particular, conventional stylet usage, assemblies and arrangements form the stylet from wire or other rigid material, which is inserted within a flexible catheter or other sleeve member and used for manipulating and guiding advancement of the flexible sleeve within a portion of the body. Opposite usage and arrangements of a stylet and introducer sleeve versus conventional devices can provide a variety of beneficial options and potential features for placement of medical device within a wound, and drain placement in particular, according to aspects, features and concepts described herein.

With continued reference to FIGS. 3A to 3C, the elongate introducer 215 is formed as an insert assembly of the stylet 220 within the sleeve 260 can include the stylet arranged as a flexible stylet inserted within a rigid sleeve. The use of a more flexible or resilient material for the stylet 220 compared with the sleeve 260 can permit user adjustment for a contour or bend of the elongate introducer as described above along with FIGS. 1A and 1B, which can modify an obtuse angle, β, formed between proximal and distal end regions of the elongate introducer. As such, the sleeve 260 can be made from a rigid, yet malleable material for permitting contour adjustments, and the stylet 220 can be made from a more flexible or resilient material that can accommodate sleeve contour adjustments while retaining stylet functionality including removability for withdrawing the stylet from within the sleeve hollow interior.

A resilient, flexible stylet in combination with a rigid sleeve can provide an effective and beneficial arrangement for various insert-type elongate introducer arrangements including elongate introducer 215 for the current example along with other arrangements including further examples shown and described below. For instance, a rigid sleeve made from aluminum or another medical grade metal or similarly stiff material can allow for thin cannular wall arrangements having high structural strength for enabling effective manipulation and advancement through a wound.

Such a sleeve arrangement can also form a shell having a hollow interior volume substantially the same as the elongate introducer for enabling various stylet arrangements, functional options, and benefits, which can be tailored according to intended usage, wound size and shape, drain details and other needs. These options can include forming the blunt tip 211 for the elongate introducer from either the rigid sleeve or from a distal end of the stylet extending through the sleeve as appropriate for the size, intended usage, or other features. In addition, as shown and described below along with other example implementations, these options can include expanded options and functionality, such as embedded scalpel components and inline incision-making functionality for the elongate introducer.

For the elongate introducer 215 shown in FIGS. 3A to 3C, the stylet 220 has a blunt tip 223 arranged to extend through an axial opening defined at the distal end region of the sleeve in the installed condition. In such an arrangement, the stylet can be made from a relatively flexible, resilient material and form blunt tip having a small radius of curvature at the tip and small diameter, such as 3 mm or less. A relatively flexible, resilient material, such as a medical grade elastomer, can form a highly effective blunt tip for readily advancing with soft tissue within a wound without damaging the soft tissue or significant risk of puncture or other internal damage while traversing the wound. Whereas formation of the blunt tip from metal or other rigid, less flexible material as appropriate for ensuring sufficient structural integrity and related functionality provided by the sleeve can increase the risk and likelihood of damage to soft tissue during use.

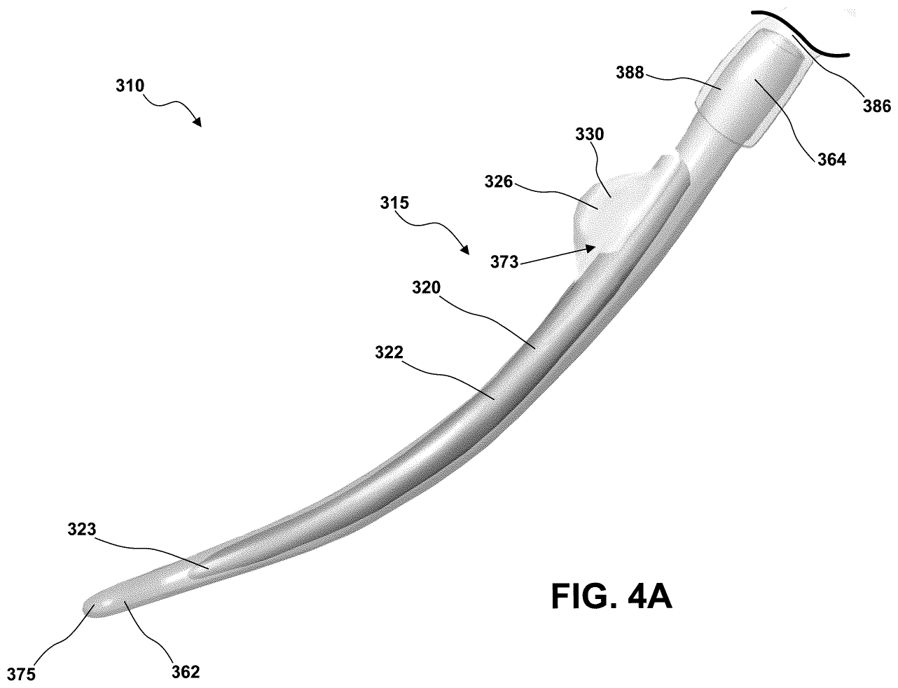

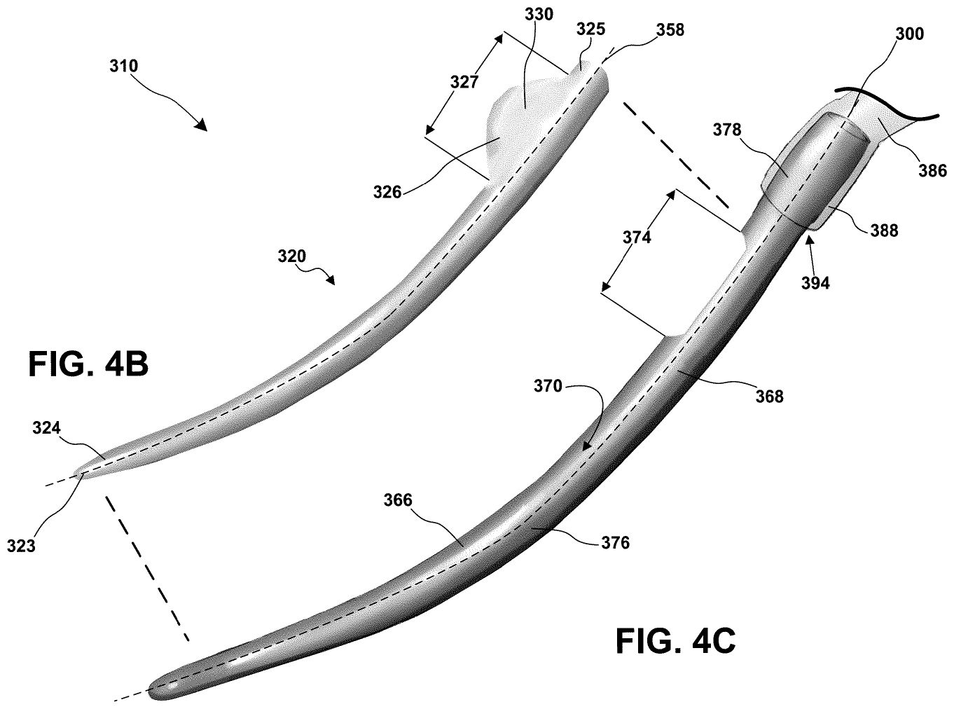

Referring now to FIGS. 4A to 4C, an introducer assembly 310 is shown generally having the same aspects and features described above along with introducer assembly 210 except as described hereafter. As such, like numbers refer to like features. Introducer assembly 310 can similarly include a flexible catheter 386 (partially shown) attached to a proximal end of a sleeve member of the elongate introducer as discussed along with FIGS. 3A to 3C for introducer assembly 210, and a fenestrated drain (not shown) coaxially attached to the flexible catheter as described above along with FIGS. 1A and 1B. Similar to introducer assembly 210, introducer assembly 310 is formed as a removable assembly of two primary components; Namely, a sleeve 360 defining a hollow interior 370, and a stylet 320 arranged as a removable insert within the hollow interior that has a laterally extending handle 326 at a proximal end region 325 of the stylet that can act as a control tab 330 during installation of the introducer assembly through a wound 12.

Introducer assembly 310 primarily differs from introducer assembly 210 described above in that that the blunt tip 311 of the introducer assembly is formed as a closed end at the distal end region of the sleeve rather than defining an axial opening at the distal end of the sleeve for the stylet to extend therethrough. The stylet 320 can include a blunt tip 323 formed at its distal end region 324, which can aid with insertion of the stylet within the sleeve through the stylet access opening 373 and for closely conforming with the shape of the sleeve 360. As such, the stylet 320 can substantially extend along the length of the hollow interior for ensuring close correspondence of maneuverability and control movements exerted on either the stylet 320 via control tab 330 or on sleeve 360 being applied to the assembly, but without the stylet extending through the distal end region of the sleeve.

As discussed above along with introducer assembly 210, such an optional arrangement for the insert stylet and sleeve assembly in which the stylet has a flexible arrangement in comparison with the sleeve having a more rigid arrangement, formation of the blunt tip 311 as part of the sleeve can provide benefits according to intended usage, wound shape, length, and disposition, and/or other factors. For instance, when the use of larger catheter sizes and corresponding drain diameters may be appropriate along with larger size and diameter for the blunt tip and elongate introducer, the likelihood of soft tissue damage may be negligible between blunt tip implementations formed from a flexible material versus a rigid material. As such, it can be beneficial to form the elongate introducer blunt tip 311 as part of the distal end region of the sleeve instead having a more complex assembly that includes extending the stylet through the sleeve. Further, for installation and placement of the drain within a wound having obstructions or anticipated interference, such as for an installation path through fascia or involving mesh within the wound, it may be beneficial for the elongate introducer 315 to have a closed distal end and blunt tip, such as formed by the sleeve.

### Lateral, Proximally-Removable Handle With 3-D Orientation/Control

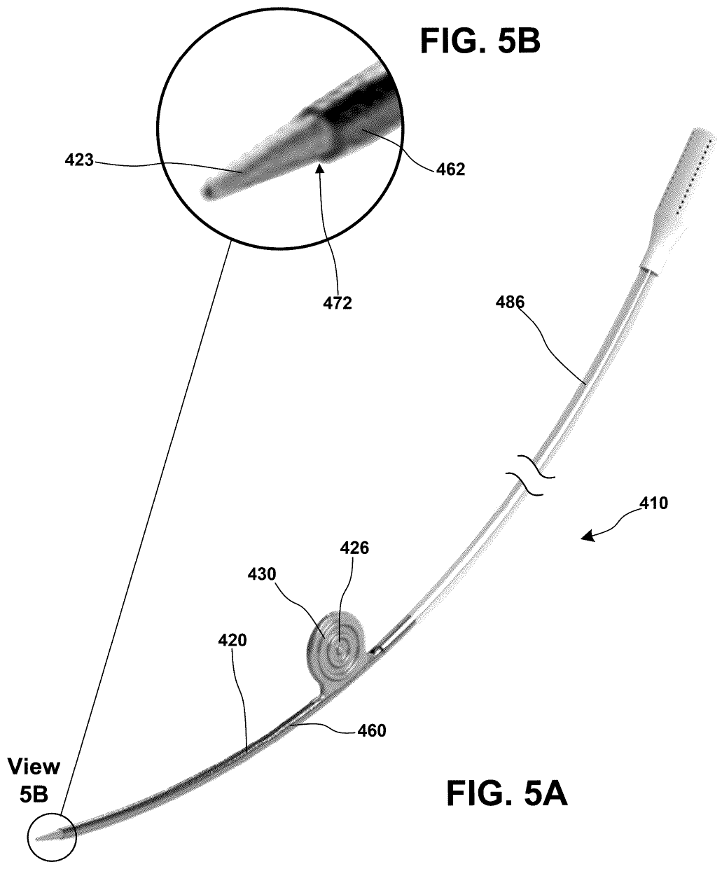

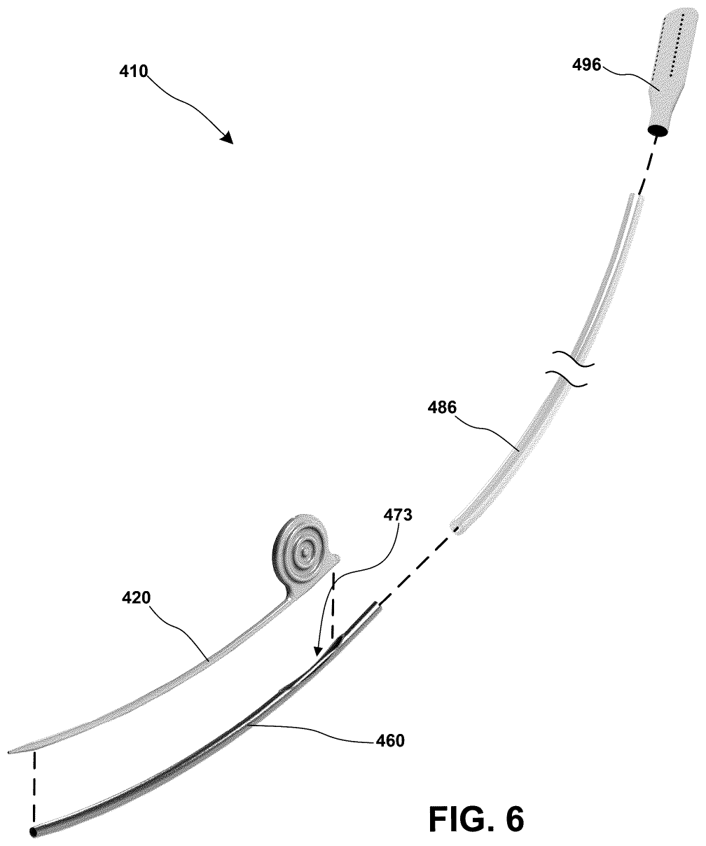

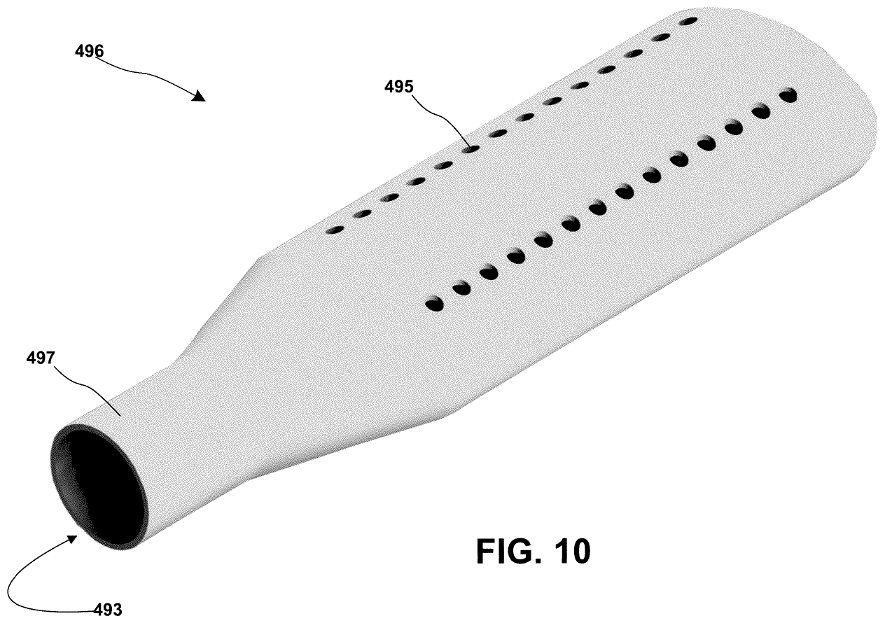

Referring now to FIGS. 5A to 10, an introducer assembly 410 is shown generally having the same aspects, features and preferences described above along with introducer assembly 210 and introducer 310 except as described hereafter. As such, like numbers refer to like features. Accordingly, introducer assembly 410 includes an elongate introducer 415 formed as an insert assembly of a stylet 420 within a sleeve 460, in which the stylet has a handle 426 arranged as a control tab 430 laterally extending from the corresponding stylet body 422 and the stylet can be removed through a stylet access opening 473 defined through the canular wall 468 of the sleeve at its proximal end region 464. Further, the stylet 420 is formed from a flexible material and the sleeve 460 is formed from a rigid material, such as a thin-walled metal shell. Similar to introducer assembly 210, the sleeve 460 defines an axial opening 472 through its distal end region 462 through which a blunt tip 423 extends to form the blunt tip 411 of the introducer assembly.

Introducer assembly 410 as depicted for the example schematic arrangement shown is generally more streamlined in comparison with the introducer assembly 210 described above. However, the arrangement of introducer assembly 410 primarily differs from introducer assembly 210 regarding the shape, configuration and functional benefits that can be provided through design options available for the lateral handle 426 and implementations for the control handle 430. Such options, implementations and related functional benefits can be provided based on the insert assembly of the flexible stylet 420 within the rigid sleeve 460 as discussed above, in which the handle 426/control tab 430 extends laterally outward from the proximal end region 464 of the sleeve extends through the stylet access opening 473 such that the stylet can be removed via the stylet access opening after substantially manipulating the elongate introducer through the wound.

Various options, implementations and related functional benefits can further be provided based on the arrangement of flexible stylet 420, which can be formed or assembled using medical grade plastics or elastomers, metals and other materials and combinations. Flexible stylet 420 and other arrangements including options shown and described hereafter can include various customized designs and options including arrangements with different features and functional benefits. Such a range of design options and features are enabled via inventive aspects, features and concepts pertaining to the use of an introducer assembly having a resilient stylet arranged as an insert within a rigid introducer sleeve, such that the stylet can be laterally and proximally withdrawn from the assembly after tunneling through a desired wound pathway and prior to advancing the catheter into the wound. Selection of a resilient or flexible stylet insert differs from conventional medical devices and uses, which make use of rigid or wire-based stylets inserted to flexible catheters or other flexible members for guiding the same, and which are axially inserted and removed.

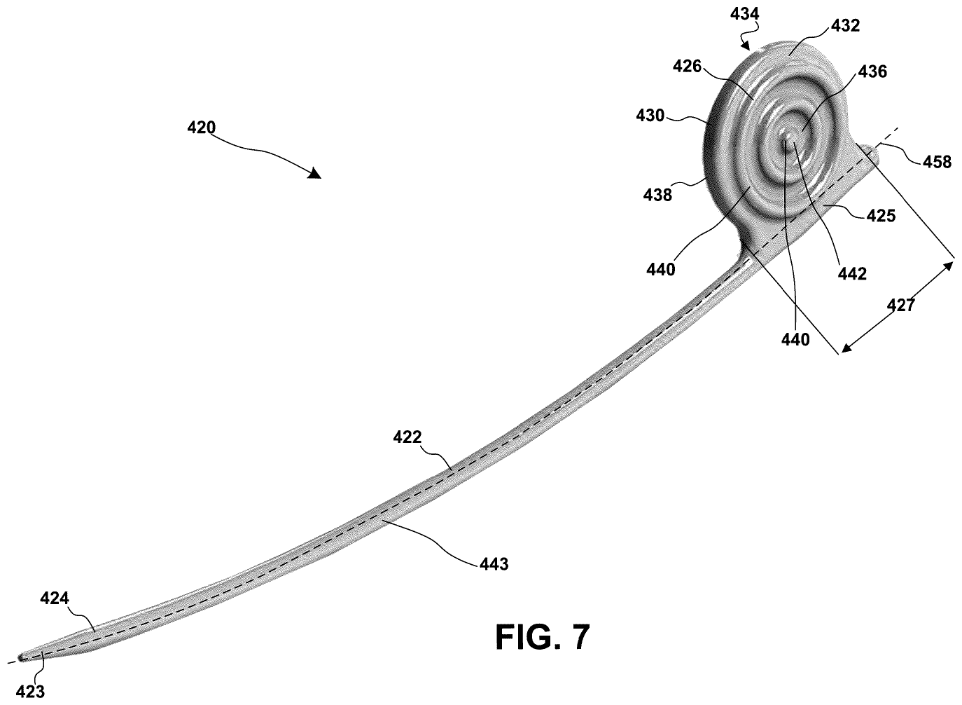

Accordingly, such a flexible stylet can be formed using a wide range of options that can allow for custom designs and expanded functionality. For instance, stylet 420 can be formed via injection molding that can enable various shape features and designs for the handle 426, such that the handle can be arranged as a control tab 430 that provides enhanced manipulation and control for the user when gripped by the single hand. As best seen in FIG. 7, control tab can be arranged for enabling three-dimensional (3D) manipulation and control by the user when gripped between the thumb and forefinger of the hand. In the arrangement shown, the control tab 430 is generally formed in a circular, disc-like shape having a first side surface 432, an opposite second side surface 434, and a relatively thin edge portion 438 therebetween. Each of the first and second sides 432,434 have a central region 436 on which a pivot feature 440 is formed. For the example of FIG. 7, the pivot feature 440 includes at least one projection 442 on each opposite side, such as central ‘bump’ or tab. When the opposing pair of bumps or tabs are gripped between the thumb and finger of the hand, the user can easily rotate or orient the elongate introducer about the pair of bumps or tabs.

As further shown in FIG. 7, the pivot feature 440 on each of the opposite first and second side surfaces 432,434 can include at least one circular rib 441. Opposite pairs of circular ribs 441 either alone or together with an opposite pair of central bumps or tab projections 441 can increase the user's grip on the control tab 430 in general due to increased roughness along with enhancing orientation control for rotating the elongate introducer 415 as needed about the central regions 436 of each side of the control tab. A plurality of concentric circular ribs 441 on each of the opposite side surfaces 434,436 can further increase overall grip with the control tab and orientation control about the central regions. In addition, the circular, disc-like shape of the control tab 430 can significantly increase the user's ability to rotate the elongate introducer 415 about its longitudinal axis proximate the control tab 430. As such, with a single hand the user can firmly grip and control the control tab 430 and control orientation of the elongate introducer 415 in three dimensions. Overall manipulation is enhanced further due to contour of sleeve 460 and elongate introducer 415 along its length such that an obtuse angle is formed between its proximal and distal end regions as discussed above along with FIGS. 1A and 1B. Accordingly, the single hand of the user can firmly grip the control tab 430 based tactile features of opposing surfaces of the tab, as well as control orientation of the elongate introducer 415 for advancing it within the wound.

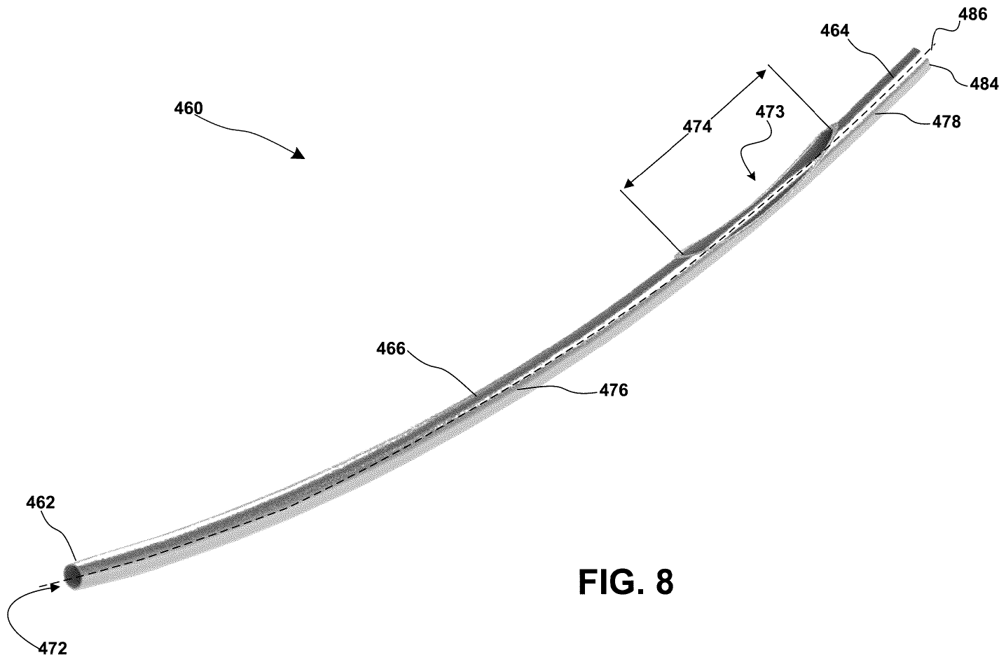

As additionally shown in FIG. 8 along with FIG. 5A, the sleeve 460 can include a rear extension 478 located proximally between the stylet access opening 473 and proximal end of the elongate introducer at the proximal end region 464 of the sleeve. The single hand of the user can grip the rear extension 478 as discussed above along with FIGS. 1A and 1B along with gripping the control tab 430 for full manipulation and control of the elongate introducer 415 including applying advancement force for tunneling through the wound and traversing the desired pathway. The distal region of the edge portion 438 for the control tab 430 with its circular, disc-like shape further provide a recess against which the single hand of the user can apply distal force for advancing the elongate introducer 415 with the wound. Thus, in the assembled condition with the flexible stylet 420 inserted within the hollow interior 470 of the sleeve 460, the example arrangement for elongate introducer 415 enables a high level of firm grip and full control of the elongate introducer for accurately manipulating it and advancing it through the wound along the desired pathway for placing the drain.

Further, after the elongate introducer 415 has been manipulated and advanced along the pathway to the desired location of the exit incision, and after the exit incision has been made with the blunt tip 411,423 advanced therethrough to extend outside the wound, the stylet 420 can easily be withdrawn from its inserted position within the sleeve 460. The user can simply pull the control tab 430 in the proximal and lateral directions with respect to the patient and wound (e.g., rearward away from the wound and upwards or sideways). Once the stylet 420 has been withdrawn, the user readily advance the sleeve 460 along with the flexible catheter 486 and fenestrated drain 496 coaxially attached therewith through the wound along the established pathway until the fenestrated drain is in place within the wound. As such, the stylet 420 while in the assembled condition with the sleeve enables high levels of grip, orientation and advancement control by a single hand of the user for tunneling the elongate introducer 415 as desired through the wound, and thereafter upon lateral and proximal withdrawal and removal of the stylet from the sleeve, the catheter and drain can easily be pulled through the wound for accurate and efficient drain placement.

It is understood that various different control tab designs and other manipulation and grip arrangements can be implemented for enhancing grip, manipulation and tunneling or advancement controls by a single hand of a user via a lateral handle or control tab of an insert stylus according to aspects and features described for stylus 420 and other example arrangements herein. Further, it is contemplated that additional options, features and attachments can be coupled with or arranged to cooperate with arrangements of an insert stylus for an introducer sleeve, such as a pull tab or other aid for assisting with removing the stylus during a procedure.

In addition, a range of options can be implemented for both the stylus and the sleeve, which can supplement functionality and/or address user or procedural preferences like whether the stylet extends fully through the sleeve and forms the blunt tip for the assembly or is fully embedded within the sleeve as described above for FIGS. 3A to 3C vs. FIGS. 4A to 4C. Enhanced features can be included as part of an elongate introducer having a stylet insert/sleeve arrangement as optional features, and/or generally incorporated for further enhancing device functionality and improving its use during related procedures, such as incorporating functionality for cutting the input and output incisions at the wound.

### Embedded Scalpel Blade/Retractable Sheath

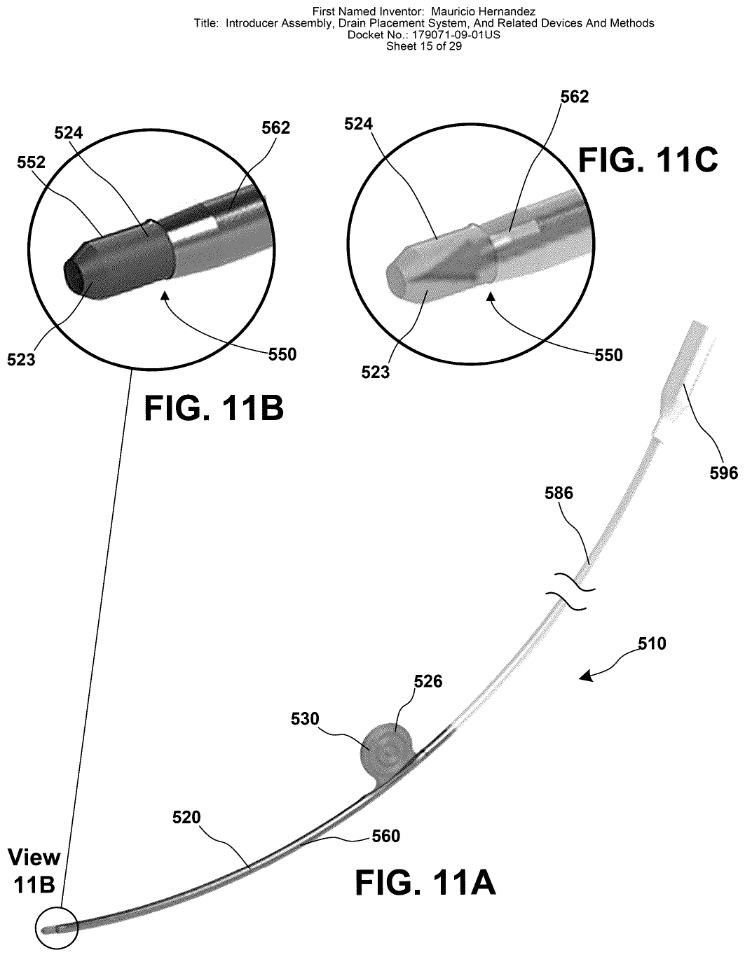

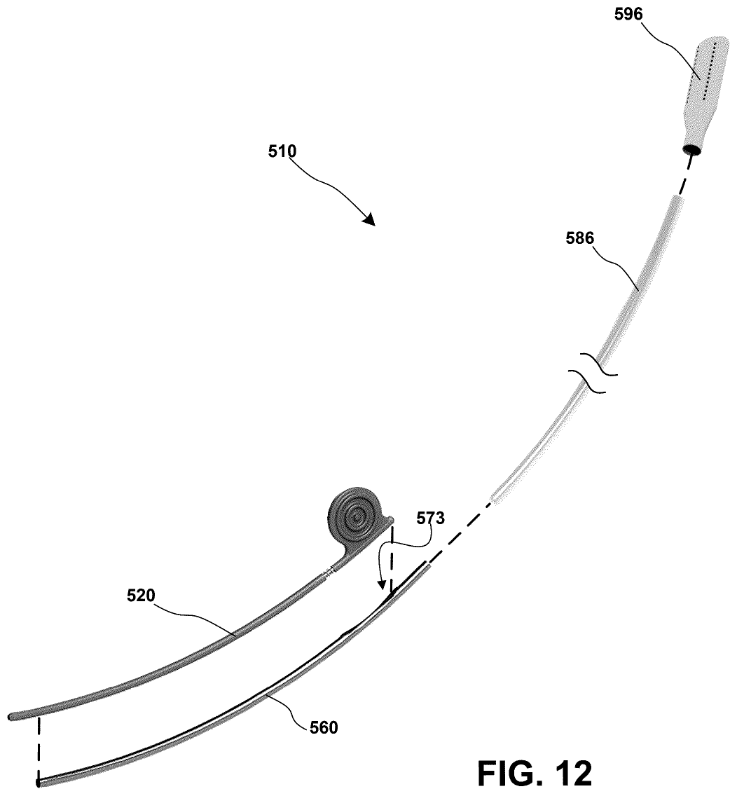

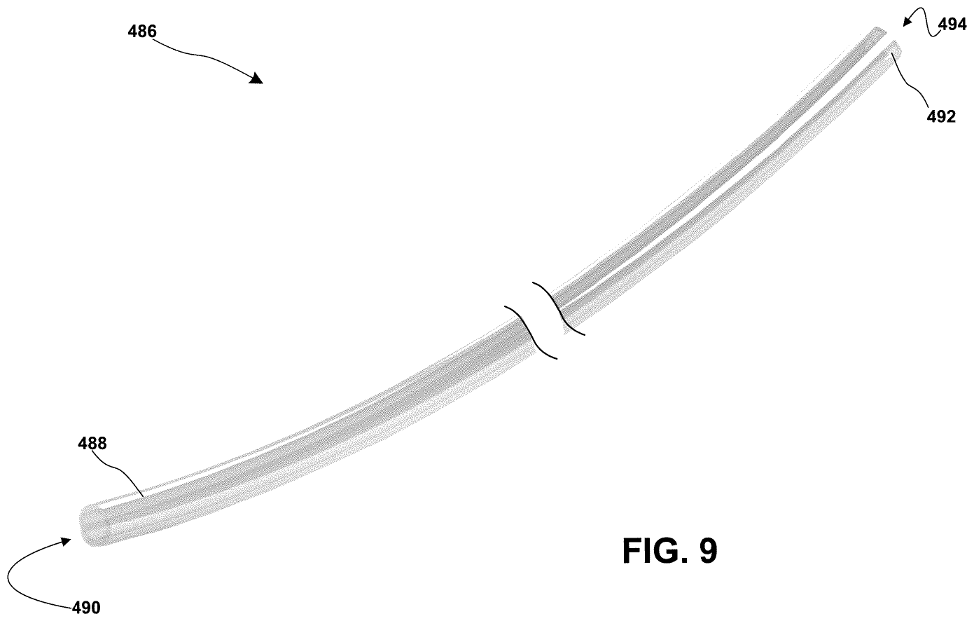

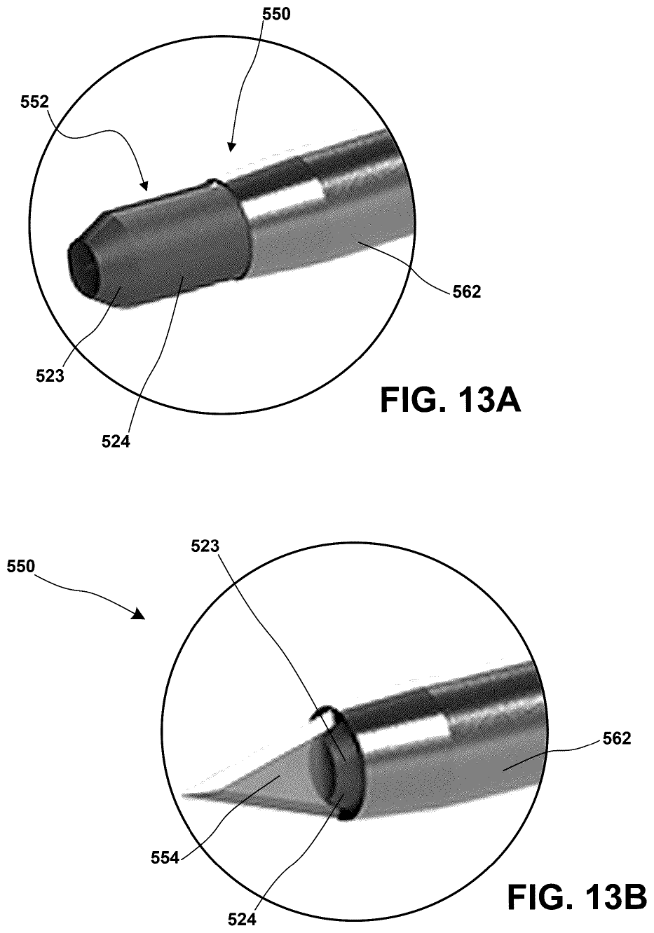

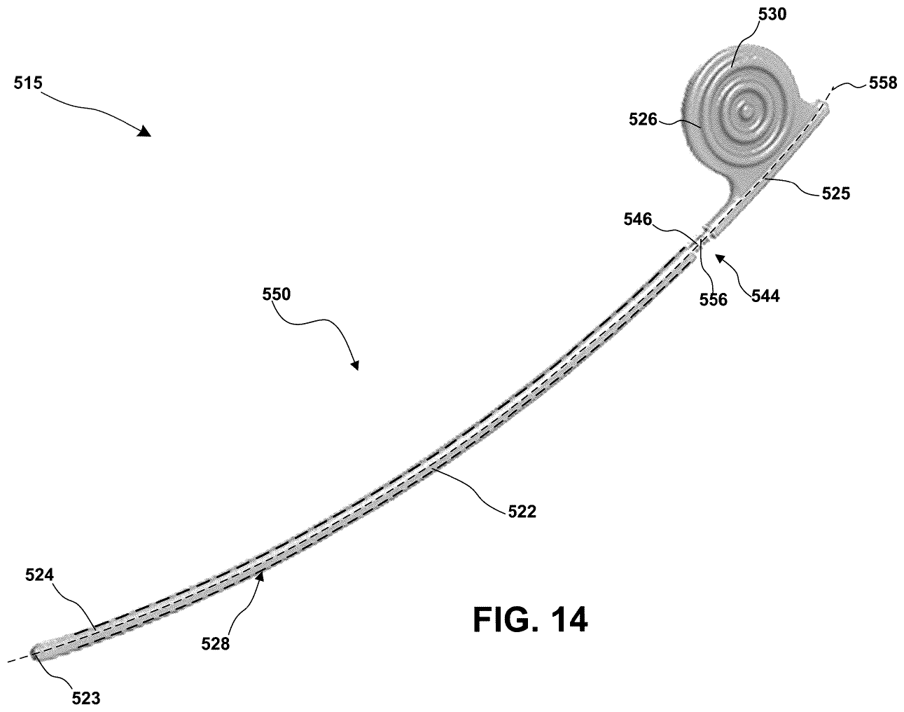

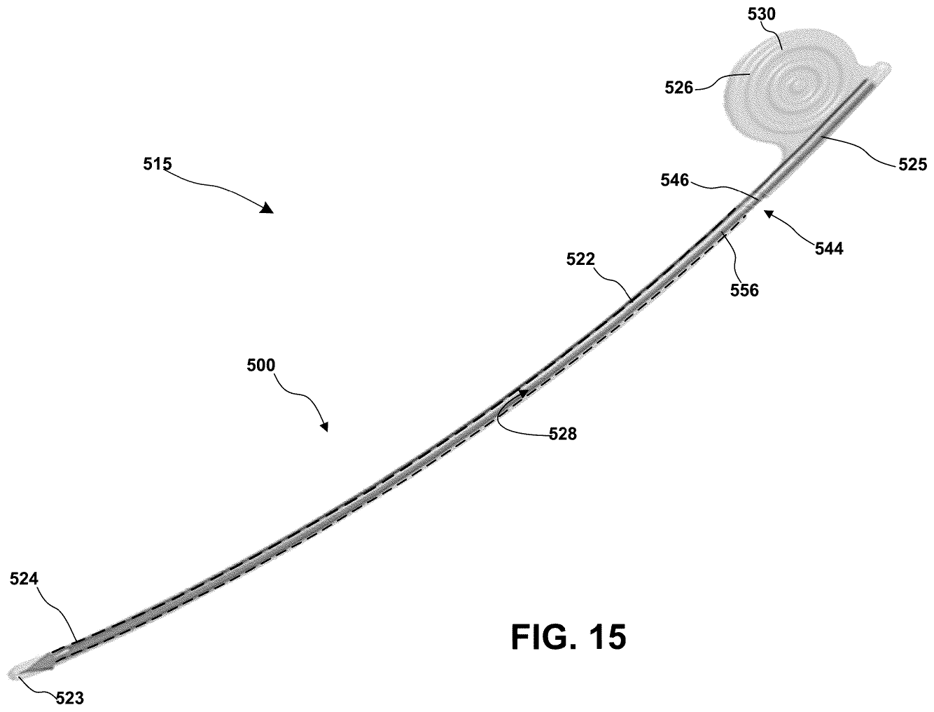

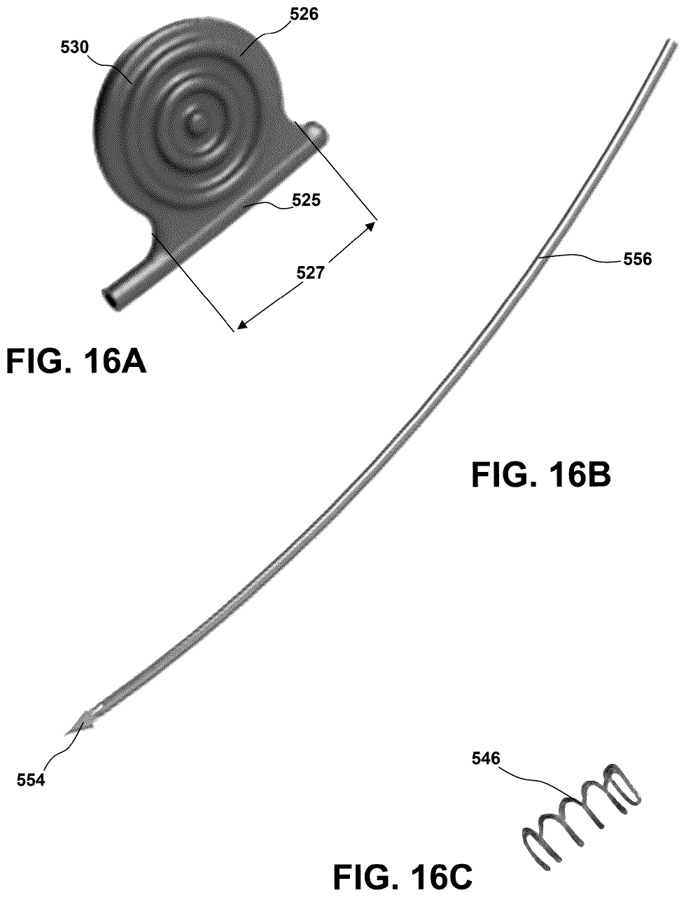

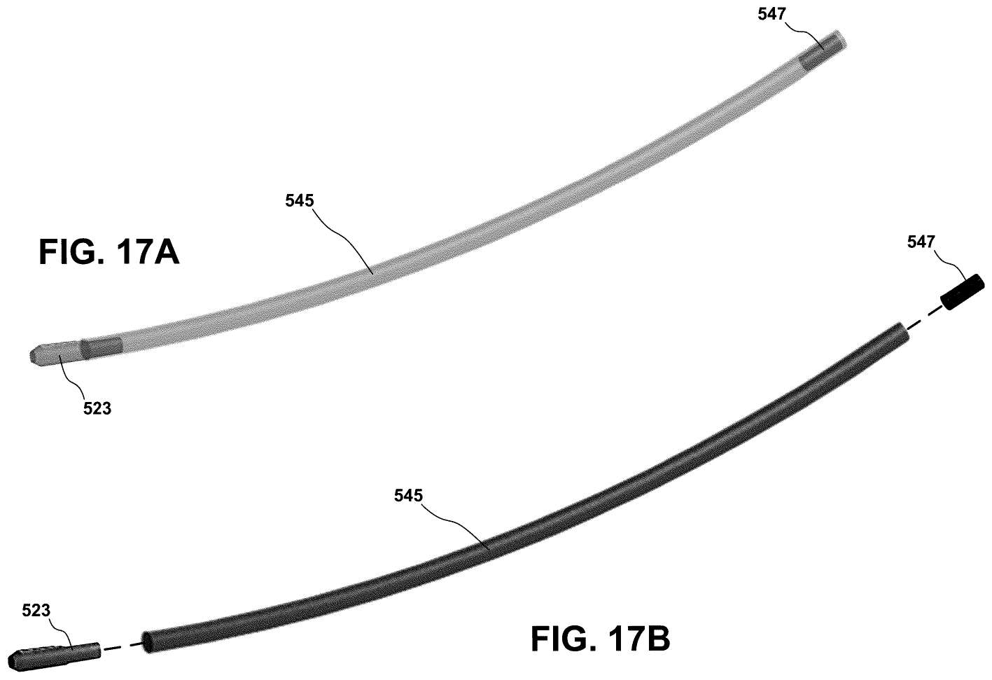

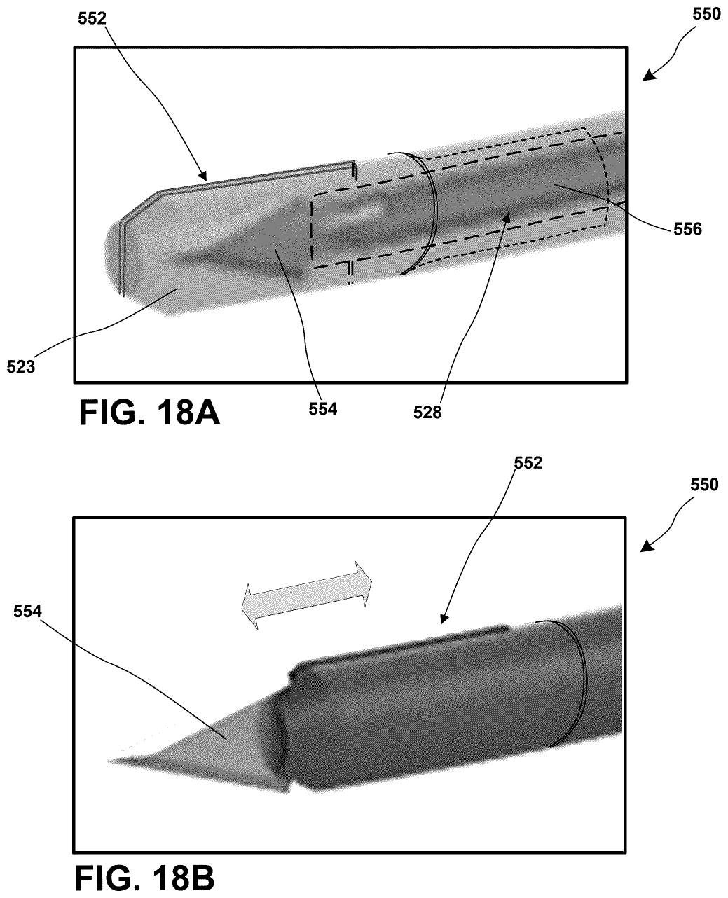

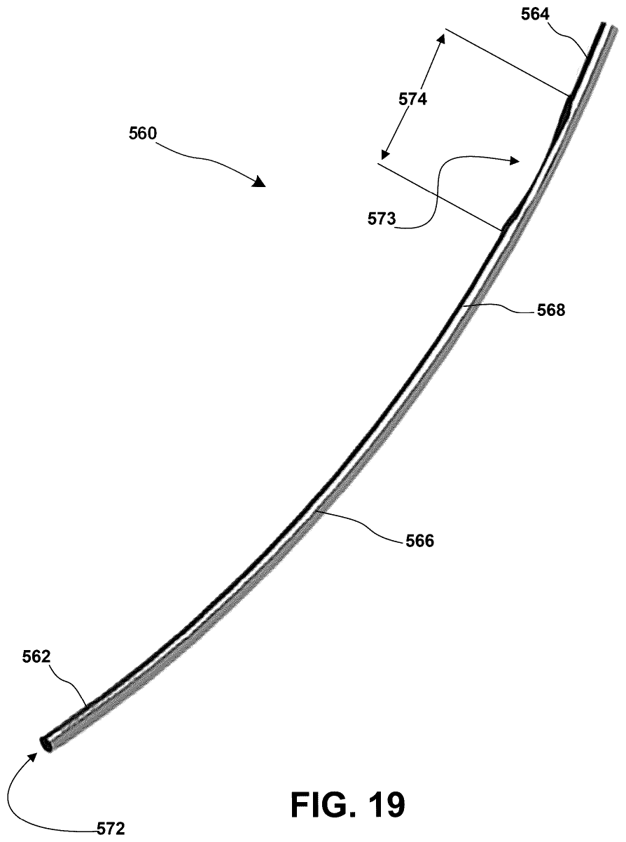

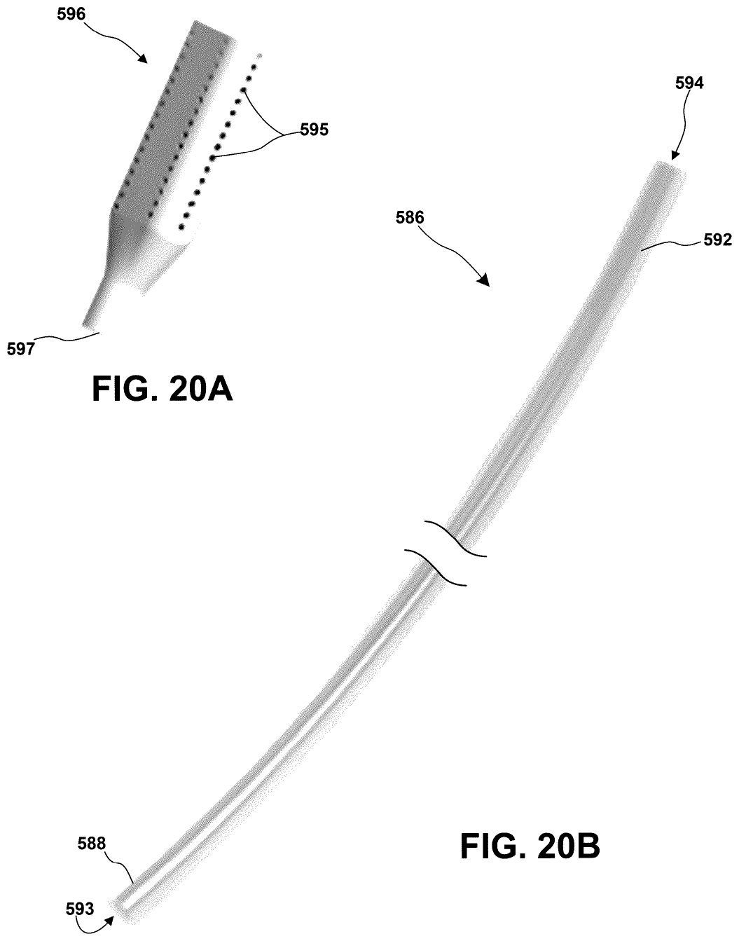

Referring now to FIGS. 11A to 20B, an introducer assembly 510 is shown that generally includes the same aspects and features described above for introducer assembly 410 except as described hereafter. As such, like numbers refer to like features. Accordingly, introducer assembly 510 includes an elongate introducer 515 formed as an insert assembly of a stylet 520 within a sleeve 560, in which the stylet has a handle 526 arranged as a control tab 530 laterally extending from the corresponding stylet body 522 and the stylet can be removed through a stylet access opening 573 defined through the canular wall 568 of the sleeve at its proximal end region 564. Further, the stylet 520 is formed from a flexible material and the sleeve 560 is formed from a rigid material, such as a thin-walled metal shell. Similar to introducer assembly 410, the sleeve 560 defines an axial opening 572 through its distal end region 562 through which a blunt tip 523 extends to form the blunt tip 511 of the introducer assembly.

Introducer assembly 510 primarily differs from introducer assembly 410 regarding insert stylet 510, which is formed as an assembly that includes an embedded scalpel blade 554 within the distal end region 524 of the stylet and the blunt tip 523. The scalpel blade 554 is located at the axial opening 572 formed in the distal end region of the sleeve body 566 outside of the sleeve body and hollow interior 570 in alignment with the longitudinal axis 558 of the stylet facing in the forward, distal direction. A slot 552 is formed in the stylet body oriented parallel with the longitudinal axis 558 and the scalpel blade 554. The scalpel blade height generally matches the diameter of the stylet and is arranged to cut both the input incision and the exit incision into and out of the wound for desired pathway. The distal end region 524 of the stylet body 522 along with the blunt tip 523 form a protective sheath 550 about the scalpel blade 554.

The stylet body protective sheath 550 can be arranged as a retractable sheath, such that the sheath retracts proximally into the sleeve for exposing the scalpel blade when cutting the entry incision and the exit incision. A scalpel support arm 556 is attached to a proximal rear side of the scalpel blade 554, which extends through a coaxial channel 528 defined longitudinally through the stylet body 522 and fixedly embedded within or attached to the proximal end region 525 of the stylet body 522. The coaxial channel 528 can be sized and arranged to translate freely in a longitudinal direction about scalpel support arm 556. A gap is defined between the proximal end region 525 of the stylet body to which the scalpel support arm 556 is attached and the distal end region 524 of the stylet body along a middle region 543 of the stylet body (shown at a distal side of the handle 526). A bias member 546, such as a spring or compressible tube member, is disposed within the gap that separates the proximal end region 525 of the stylet body 522 from the distal end region 524 at the middle region 543.

The bias member 546 biases the protective sheath 550 in the distal direction, such that the blunt tip 523 covers the scalpel blade 554 for tunneling and advancement within the wound. The spring strength of the bias member 546 maintains the protective sheath 550 in its default, forward or distal position covering the scalpel blade. The spring strength can further be arranged for allowing the protective sheath 550 to retract in the proximal or rearward direction when the blunt tip 523 makes interfering contact with the patient's skin at both sides of the wound (i.e., input incision location and exit incision location). In response to interfering contact with the patient's skin, the protective sheath 550 is arranged to retract proximally about the scalpel blade into the hollow interior 570 of the sleeve, which exposes the scalpel blade such that it contacts the skin and makes the incision for entry into or exit from the wound.

Such an arrangement can greatly simplify the wound placement procedure by incorporating incision cutting into a stream-lined procedure for traversing a desired pathway through the wound. Doing so can allow the user to maintain contact and control of the elongate introducer 515 with the single hand from the start of its advancement into the wound through advancement of the blunt tip 523 through the wound and out of the exit incision. Thereafter, the user can remove the stylet 520 from the sleeve 560 without having to stop in the interim, and can thereafter pull the sleeve 560 and attached catheter and fenestrated drain through the wound for drain placement. The use of a pre-sized scalpel blade 554 embedded within the elongate introducer 515 can ensure the incisions are cut appropriately for corresponding well with the size of the elongate introducer and catheter along with forming a tight fit about the catheter at the exit incision for sealing the drain within the wound.

### Manually Retractable Sheath

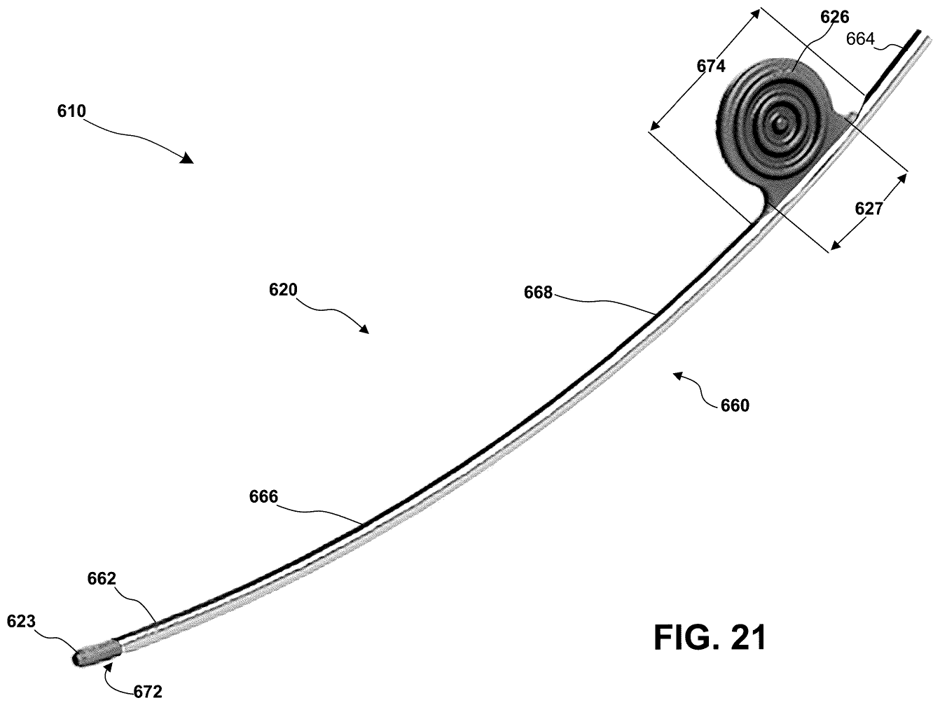

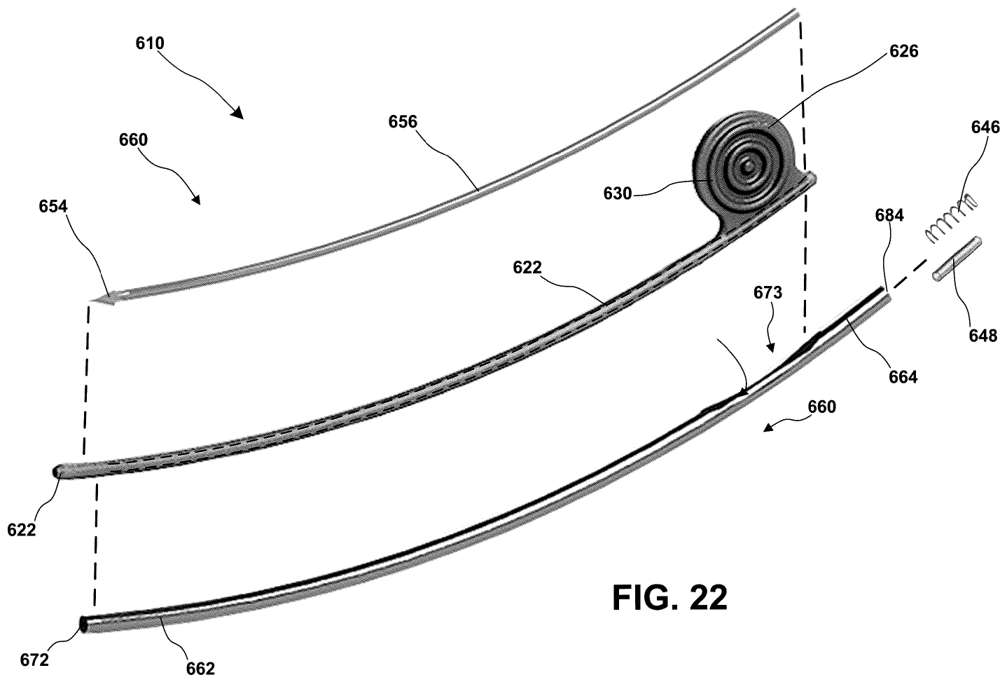

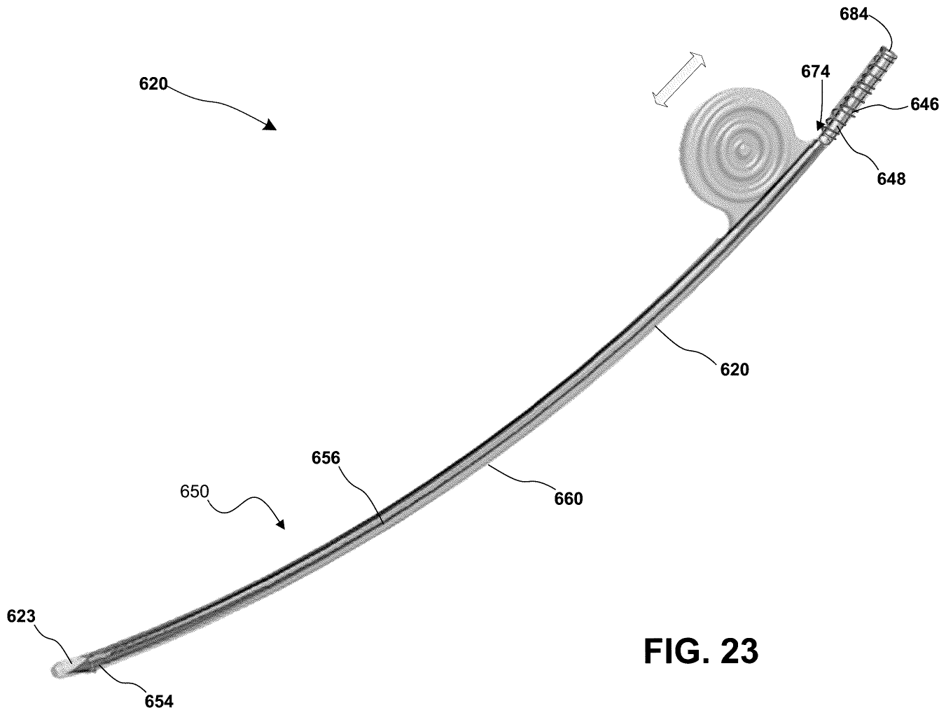

Referring now to FIGS. 21 to 23, an introducer assembly 610 is shown that generally includes the same aspects and features described above for introducer assembly 510 except as described hereafter. As such, like numbers refer to like features. Accordingly, introducer assembly 610 includes an elongate introducer 615 formed as an insert assembly of a stylet 620 within a sleeve 660, in which the stylet has a handle 626 arranged as a control tab 630 laterally extending from the corresponding stylet body 622 such that the stylet can be removed through a stylet access opening 673 defined through the canular wall 668 of the sleeve at its proximal end region 664. Further, the stylet 620 is formed from a flexible material and the sleeve 660 is formed from a rigid material, such as a thin-walled metal shell. Similar to introducer assembly 410 and 510, the sleeve 660 defines an axial opening 672 through its distal end region 662 through which a blunt tip 623 extends to form the blunt tip 611 of the introducer assembly.

Introducer assembly 610 primarily differs from introducer assembly 510 regarding the arrangement of the protective sheath 650, which is arranged as manually retractable protective sheath. As such, the coaxial channel 628 defined through the stylet body 623 extends proximally from a rear side of the scalpel blade 654 through the middle region 643 and the proximal end region 525 of the stylet body slightly beyond a proximal end of the stylet, and stylet body is formed as a unitary member without a gap. Accordingly, the entire length of the stylet body 623 can be arranged to translate freely in the proximal direction about the scalpel support arm 656 located within the coaxial channel 628. The bias member 646 is located within the rear extension 678 of the sleeve 660 within a portion of the hollow interior 670 within the rear extension. As shown, the sleeve has closed proximal end such that the hollow interior is closed at a proximal end of the sleeve. Note that sleeve 560 of introducer assembly 510 can also be closed, but can also have an axial opening defined in the proximal end.

A spring arm stop 648 is further disposed coaxially within the bias member 546. Further, the stylet access opening 673 has an access opening length 674 that is larger than a handle length 627, which permits the user to slide the handle 626 and thereby the stylet body 622 longitudinally rearward or proximally. When the stylet body 622 translates rearward, a proximal end of the scalpel support arm 656 is blocked from sliding by the scalpel arm stop 648 coaxially disposed within the bias member 646 for retaining the scalpel blade in place while the protective sheath 650 is retracted due to rearward translation of the stylet body 622.

As such, the stylet 620 in the assembled condition within the sleeve 660 forms a manually retractable protective 650, which the user can retract as required for making the input and exit incisions and release thereafter. The compressed bias member 646 biases the stylet body 622 distally, such that when the user releases the handle 626 the stylet body 622 slides distally to its default position responsive to restoring force from the bias member and again protects the scalpel blade 654 with the blunt tip 623 fully advanced to its forward distal position.

### Method/Placement Procedure

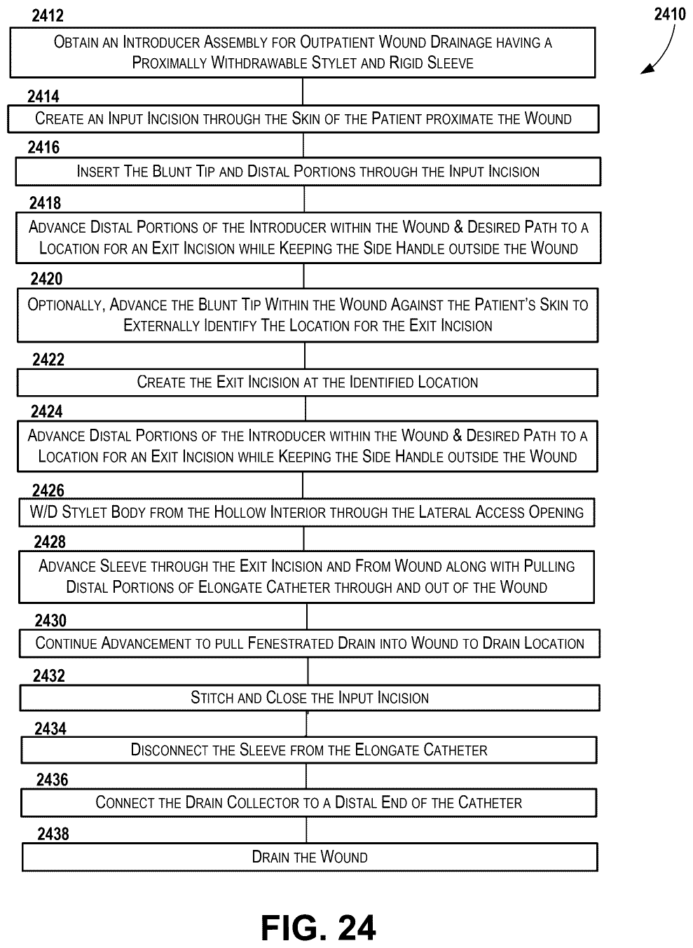

Referring now to FIG. 24, a Method 2410 is generally depicted for placing a drain and attached catheter or other medical device at a desired location within a wound using an introducer assembly according to aspects, features and concepts described herein, such as introducer assembly 410, 510 or 610 described above. Method 2410 generally includes aspects and features described above along with FIGS. 2A to 2F except as discussed hereafter and with respect to aspects and features shown and described above for introducer assemblies 410, 510 or 610 that differ from introducer assembly 110 used with the procedure depicted in FIGS. 2A to 2F.

As shown in FIG. 24, Method 2410 includes 2412 Obtaining an introducer assembly for outpatient wound drainage having a proximally withdrawable stylet and rigid sleeve, 2414 Creating an input incision through the skin of the patient proximate the wound, 2416 Inserting the blunt tip and distal portions through the input incision, and 2418 Advancing distal portions of the introducer within the wound and desired path to a location for an exit incision while keeping the side handle outside the wound. Similar to the procedure of FIGS. 2A to 2F, Method 2410 can optionally include 2420 Advancing the blunt tip within the wound against the patient's skin to externally identify the location for the exit incision. Method 2410 can further include 2422 Creating the exit incision at the identified location, and 2424 Advancing distal portions of the introducer assembly within the wound and desired path to a location for the exit incision while keeping the side handle outside the wound.

The Method 2410 can further include 2426 Withdrawing the stylet body from the hollow interior through the lateral access opening of the sleeve, 2428 Advancing the sleeve through the exit incision and from the wound along with pulling distal portions of the elongate catheter through and out of the wound, and 2430 Continuing Advancement to pull the fenestrated drain into the wound and to the desired drain location. In addition, Method 2410 can include 2432 Stitching and Closing the input incision, 2434 Disconnecting the sleeve from the elongate catheter, 2436 Connecting the drain collector to a distal end of the catheter, and 2438 Draining the wound.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

| Ref. #s | Description | Group |
|---|---|---|
| x10 | Introducer Assembly | Assembly |
| x11 | Blunt Tip (assy) | Assembly |
| x12 | Wound | Method |
| x14 | Entry incision | Method |
| x15 | Elongate Introducer | Assembly |
| x16 | Exit incision | Method |
| x18 | Hand | Method |
| x20 | Stylet | Stylet |
| x22 | Stylet Body | Stylet |
| x23 | Blunt Tip | Stylet |
| x24 | Distal end region | Stylet |
| x25 | Proximal end region | Stylet |
| x26 | Handle | Stylet |
| x27 | Handle length | Stylet |
| x28 | Coaxial channel | Stylet |
| x30 | Control tab | Stylet |
| x32 | First side surface | Stylet |
| x34 | Second side surface | Stylet |
| x36 | Central region | Stylet |
| x38 | Edge portion | Stylet |
| x40 | Pivot feature | Stylet |
| x41 | Circular rib | Stylet |
| x42 | Projection | Stylet |
| x43 | Middle region | Stylet |
| x44 | Retraction gap | Stylet |
| x45 | Sheath body | Stylet |
| x46 | Bias member | Stylet |
| x47 | Spring stop | Stylet |
| x48 | Scalpel arm stop | Stylet |
| x50 | Retractable Sheath | Stylet |
| x52 | Slot | Stylet |
| x54 | Scalpel blade | Stylet |
| x56 | Scalpel support arm | Stylet |
| x58 | Longitudinal axis | Stylet |
| x60 | Sleeve | Sleeve |
| x62 | Distal end region | Sleeve |
| x64 | Proximal end region | Sleeve |
| x66 | Sleeve body | Sleeve |
| x68 | Canular wall | Sleeve |
| x70 | Hollow interior | Sleeve |
| x72 | Axial opening | Sleeve |
| x73 | Stylet access opening | Sleeve |
| x74 | Access opening length | Sleeve |
| x75 | Blunt tip | Sleeve |
| x76 | Middle region | Sleeve |
| x78 | Rear extension | Sleeve |
| x80 | Longitudinal axis | Sleeve |
| x82 | Obtuse angle | Sleeve |
| x84 | Closed end | Sleeve |
| x86 | Flexible Cather | Catheter |
| x88 | Distal end | Catheter |
| x90 | Axial opening (distal end) | Catheter |
| x92 | Proximal end | Catheter |
| x93 | Axial opening (DRAIN distal end) | Drain |
| x94 | Axial opening (proximal end) | Catheter |
| x95 | Drain openings | Drain |
| x96 | Fenestrated drain | Drain |
| x97 | Distal end (drain) | Drain |
| x98 | Drain collector | Collector |
| x99 | Proximal end (collector) | Collector |

What is claimed is:

1. An introducer assembly for guiding a catheter through a wound of a patient, the introducer assembly comprising an elongate introducer having a distal end, an opposite proximal end and a middle region therebetween, the introducer comprising:
a stylet comprising:
a stylet body longitudinally extending from a distal end region to an opposite proximal end region; and
a handle at the proximal end region laterally extending from the stylet body;
a sleeve having a distal end region, a proximal end region, and a sleeve body longitudinally extending therebetween, the sleeve body having a canular wall defining a hollow interior extending from at least a portion of the proximal end region to the distal end region, a stylet access opening into the hollow interior defined through a side portion of the canular wall at the proximal end region and configured to receive the stylet body for slidable insertion into and out of the hollow interior; and
a blunt tip defined at the distal end of the elongate introducer, the blunt tip formed from one of a sleeve blunt tip defined at the distal end region of the sleeve and a stylet blunt tip defined at the distal end region of the stylet extending through an axial opening defined at the distal end region of the sleeve when the stylet body is installed within the hollow interior of the sleeve;
wherein:
the stylet body is installed within the hollow interior of the sleeve;
the proximal end of the elongate introducer is configured to enable manipulation when gripped by a single hand of the user for inserting the blunt tip and a portion of the middle region of the elongate introducer into and through a wound of a patient;
the handle extends laterally outward through the stylet access opening; and
the handle is configured to enable removal of the stylet body from the hollow interior through the stylet access opening when gripped by a user while the middle region of the portion of the middle region of the elongate introducer is within the wound.

2. The introducer assembly of claim 1, wherein the handle forms a control tab configured to enhance user control and manipulation of the elongate introducer when gripped by the single hand.

3. The introducer assembly of claim 2, wherein the control tab comprises:
a first side surface;
a second side surface opposite the first side surface; and
an edge portion between the first side surface and the second side surface;
wherein:
the first side surface and the second side surface are oriented generally parallel with a longitudinal axis of the stylet body;
a central region of each of the first side surface and the second side surface are laterally offset from the longitudinal axis; and
the control tab is configured for being gripped between a thumb and a finger of the user's single hand.

4. The introducer assembly of claim 3, wherein:
the control tab is generally disc-shaped; and
the first side surface and the second side surface each define a circular shape.

5. The introducer assembly of claim 3, wherein a first pivot feature is formed on the first side surface and a second pivot feature is formed on the second side surface opposite the first side surface.

6. The introducer assembly of claim 5, wherein the first pivot feature includes a first circular rib and the second pivot feature includes a second circular rib.

7. The introducer assembly of claim 6, wherein the first circular rib comprises a plurality of first concentric ribs, and the second circular rib comprises a plurality of second concentric ribs.

8. The introducer assembly of claim 5, wherein the first pivot feature includes a first projection extending from the first side surface and the second pivot feature includes a second projection extending from the second side surface.

9. The introducer assembly of claim 2, the sleeve further comprising a rear extension at the proximal end region longitudinally extending rearward in a proximal direction away from the stylet opening.

10. The introducer assembly of claim 1, wherein a longitudinal axis of the sleeve is contoured between the distal end region and the proximal end region, such that an obtuse angle is defined between the longitudinal axis at the proximal end region and the distal end region of the sleeve.

11. The introducer assembly of claim 10, wherein:
the sleeve body is malleable permitting user adjustment of the contoured sleeve body and the obtuse angle; and
the stylet body is configured to flex according to the user adjustment of the contoured sleeve body.

12. The introducer assembly of claim 1, wherein:
the sleeve further comprises a proximal end and the cannular wall at the proximal end defines one of:
an axial opening into the hollow interior through the proximal end; and
a closed end wherein the hollow interior is sealed at the proximal end; and
the introducer assembly further comprises a flexible catheter having a distal end defining an axial opening, an opposite proximal end defining an axial opening, and an elongate tubular body defining a longitudinal pathway between the axial opening at the distal end and the axial opening at the proximal end, the distal end coaxially attached to the sleeve at the proximal end;
wherein, after withdrawal of the stylet body from the hollow interior, the sleeve is configured to advance through the wound and pull the flexible catheter into the wound.

13. The introducer assembly of claim 12, further comprising:
a fenestrated drain attached to the catheter at the proximal end in fluid communication with the longitudinal pathway through the catheter;
wherein;
after withdrawal of the stylet body from the hollow interior, the sleeve and a distal portion of the flexible catheter are configured to be pulled out of the wound and the patient to advance the fenestrated drain into the wound; and
the distal portion of the flexible catheter is configured to disconnect from the sleeve and attach to a drain collector for wound drainage.

14. The introducer assembly of claim 1, wherein the blunt tip is formed from the stylet blunt tip defined at the distal end region of the stylet extending through the axial opening defined at the distal end region of the sleeve when the stylet body is installed within the hollow interior of the sleeve, the stylet body further comprising:
a slot oriented in the longitudinal direction of the stylet body defined in the distal end region of the stylet body and through the blunt tip; and
a scalpel blade embedded in the stylet body within the slot and located outside the sleeve proximate the axial opening at the distal end region of the sleeve, the scalpel blade configured to cut an entry incision through the user's skin for gaining entry into the wound and to cut an exit incision through the user's skin for withdrawing the sleeve from the wound;
wherein:
the distal end region of the stylet body, the blunt tip, and the slot form a protective sheath about the scalpel blade; and
the protective sheath is configured to retract proximally into the sleeve for exposing the scalpel blade for cutting the entry incision and the exit incision.

15. The introducer assembly of claim 13, wherein:
the sheath is selectively retractable by the user;
the handle has a first length in the longitudinal direction of the stylet;
the stylet access opening has a second length in the longitudinal direction larger than the first length;
the stylet body and the sheath are biased forward in the distal direction in the assembled condition and are translatable rearward in the proximal direction within the hollow interior of the sleeve; and
the handle is configured to enable the user to slide the handle rearward in the proximal direction within the stylet access opening and thereby translate the stylet body and the sheath to the retracted position for exposing the scalpel blade.

16. The introducer assembly of claim 13, wherein the sheath is configured to retract automatically in response to contact interference with skin of the patient when forming the entry incision and the exit incision, the stylet body further comprising:
a coaxial channel defined through the stylet body extending between the rear proximal side of the scalpel blade to the proximal end region;
a retraction gap defined at a middle region of the stylet body;
a bias member disposed within the retraction gap distally biasing the distal end region of the style body longitudinally away from the proximal end region of the stylet body; and
a scalpel support extending from a rear proximal side of the scalpel blade through the coaxial channel, the retraction gap and the bias member to the proximal end region of the stylet body, the scalpel support fixedly attached to the proximal end region stylet body and the sheath is configured to translate about the scalpel support.

17. The introducer assembly of claim 1, wherein the sleeve is tapered between the distal end region and the proximal end region having a first cross-sectional area at the distal end region and a second cross-sectional area at the proximal end region that is larger than the first cross-sectional area.

18. An outpatient method for draining fluids from a wound of a patient, the method comprising: obtaining an introducer assembly as recited in claim 13;
creating an input incision through the skin of the patient proximate the wound;
inserting through the input incision the blunt tip and a portion of the middle region of the introducer assembly;
advancing the introducer assembly within the wound along a desired drain path from the input incision to a desired location for an exit incision while keeping the side grip outside the wound;
optionally, advancing the blunt tip within the wound against the patient's skin to identify a location for an exit incision;
creating the exit incision;
advancing the blunt tip through the exit incision;
withdrawing the stylet body from the hollow interior of the sleeve through the lateral access opening of the sleeve disposed outside of the wound;
advancing the sleeve through the exit incision and out of the wound and thereby concurrently pulling a portion of the elongate catheter into the wound through the input incision and out of the wound through the exit incision;
continue advancing the elongate catheter through and out of the wound including drawing the fenestrated drain into the wound through the input incision and through the wound until the fenestrated drain is positioned at a desired drainage location;
stitching closed the input incision;
disconnecting the proximal end region of the sleeve from the distal end of the elongate catheter;
connecting the distal end of the elongate catheter to a drain collector; and
draining the wound.

19. The outpatient method of claim 18, wherein:
the blunt tip is formed from the stylet blunt tip extending through the axial opening defined at the distal end region of the sleeve when the stylet body is installed within the hollow interior;
the stylet body includes an embedded scalpel blade and the blunt tip and the distal end region of the stylet body form a retractable sheath configured to retract rearward into the sleeve to expose the embedded scalpel blade;
creating the input incision comprises:
retracting the retractable sheath to expose the embedded scalpel blade; and
advancing the embedded scalpel blade through the patient's skin to create the input incision; and
creating the exit incision comprises:
retracting the retractable sheath to expose the embedded scalpel blade; and
advancing the embedded scalpel blade through the patient's skin to create the exit incision.

20. The outpatient method of claim 19, wherein:
the retractable sheath is selectively retractable by the user; and
for creating the input incision and creating the exit incision, each of the retracting the retractable sheath comprises:
manually retracting the retractable sheath; and
advancing the exposed scalpel blade through the user's skin.

21. The outpatient method of claim 19, wherein:
the retractable sheath is automatically retractable in response to interring contact of the sheath with the user's skin; and
for creating the input incision and creating the exit incision, each of the retracting the retractable sheath comprises:
advancing the introducer assembly for advancing the blunt tip and the sheath into contact with the user's skin; and
continuing advancing the introducer assembly for advancing the exposed scalpel into and through the user's skin.
